(19) United States
(12) Patent Application Publication
(10) Pub. No.: US 2013/0144087 A1
(43) Pub. Date: Jun. 6, 2013

(54) CO-GASIFICATION OF AQUATIC BIOMASS AND COAL

(71) Applicant: Celanese International Corporation, Irving, TX (US)

(72) Inventor: Dinesh Arora, League City, TX (US)

(73) Assignee: Celanese International Corporation, Irving, TX (US)

(21) Appl. No.: 13/691,239

(22) Filed: Nov. 30, 2012

Related U.S. Application Data
(60) Provisional application No. 61/566,215, filed on Dec. 2, 2011.

(51) Int. Cl. C01B 3/02; C07C 29/149

(52) U.S. Cl. CPC C01B 3/02; C07C 29/149
USPC 568/884; 252/373

(57) ABSTRACT

The invention also relates to co-gasification processes for forming syngas from aquatic biomass and a fossil fuel. In one aspect, the invention is to a process for producing syngas, comprising: introducing aquatic biomass, a fossil fuel, water and oxygen to a gasifier and forming syngas comprising hydrogen, carbon monoxide and carbon dioxide; and feeding aquatic biomass with carbon dioxide derived from the syngas. In other aspects, the invention relates to integrated processes for producing industrial chemicals, such as alcohols, carboxylic acids, esters, aldehydes, olefins and polymers from such syngas.

### CROSS-REFERENCE TO RELATED APPLICATION

This application claims priority to U.S. Provisional Appl. No. 61/566,215, filed Dec. 2, 2011, the entirety of which is incorporated herein by reference.

### FIELD OF THE INVENTION

The present invention relates generally to integrated processes for producing industrial chemicals such as alcohols, carboxylic acids, esters, aldehydes, olefins and polymers from biomass. In particular, the invention relates to improved co-gasification processes for forming syngas from biomass and a fossil fuel, where the syngas is optionally well suited for forming one or more industrial chemicals and, in particular, ethanol.

### BACKGROUND OF THE INVENTION

Biomass gasification is a widely known process for producing synthesis gas, commonly referred to as syngas. Almost all types of biomass may be gasified. Biomass gasification is described, for example, in U.S. Pat. Nos. 7,736,402; 6,972,114; 6,133,328; 5,666,890; 4,699,632; and 4,544,375; US Pub. Nos. 2011/0195365; 2010/0301273; 2010/0299995; 2010/0270506; 2010/0132633; 2009/0183693; and 2009/0183430; and PCT Pubs. WO2000/069994 and WO2011/097648, the entireties of which are incorporated herein by reference. Syngas is a gas mixture comprising varying amounts of carbon monoxide (CO) and hydrogen (H₂), and optionally carbon dioxide.

Under low oxygen conditions and high temperatures, biomass gasification may generate a fuel known as producer gas. Producer gas is similar to syngas but also contains nitrogen. Though syngas and/or producer gas may have a lower energy density than natural gas, syngas and/or producer gas may suitable for use in a variety of applications.

In commercial applications, syngas and/or producer gas from biomass has a usable chemical energy content and may be used in various applications, such as heat production, combined heat and power (CHP) generation, stand-alone electricity production, and in fuels. Syngas and/or producer gas, for example, may be used as a fuel for gas turbines or internal combustion engines. Currently, commercial technologies for syngas primarily include heat, power, and combined heat and power systems of comparatively small scale, e.g., less than 10 MW electric or 65 MJ/hr thermal. Exemplary biomass gasifiers for heat production, combined heat and power (CHP) generation, stand-alone electricity production, and fuels are described in U.S. Pub. Nos. 2011/104575; 2010/0251608; and 2008/0216415, the entireties of which are incorporated herein by reference.

There is also an interest in using syngas to produce industrial chemicals. However, there are several challenges in using biomass to produce syngas for integration with the production of industrial chemicals. Unlike many other syngas applications, production of industrial chemical may require a high grade syngas having a low nitrogen, argon and carbon dioxide content. In addition, due to economics, industrial chemicals may require significant capital expenditures to operate a cost-efficient biomass gasification. Because biomass gasifies at relatively low temperatures compared to coal and petroleum coke, biomass gasification may produce tars and phenolics that can foul and poison downstream catalysts and therefore require removal and costly disposal.

Owing to its increasing use in fuel applications, a particularly desired industrial chemical that may be derived ultimately from syngas is ethanol, which more commonly is produced from petrochemical feed stocks, such as oil, natural gas, or coal, or from fermentation processes. Conventional methods for producing ethanol from petrochemical feed stocks, as well as from cellulose materials, include the acid-catalyzed hydration of ethylene, methanol homologation, direct alcohol synthesis, and methanol-to-olefin (MTO) technologies. Instability in petrochemical feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol, which is suitable for fuels or human consumption. In addition, the fermentation of starchy materials competes with food sources and places restraints on the production of ethanol.

Current demand for ethanol is expected to increase because ethanol is a clean burning fuel source that may be derived from renewable sources. Although fermentation provides a production pathway from biomass to ethanol, competing uses of fermentation raw materials such as corn effectively limit the total amount of ethanol that can be produced from fermentation. Thus, there is a strong interest in producing ethanol according to renewable fuels standards using a method other than fermentation.

One proposal is to produce ethanol from algae as described in U.S. Pat. No. 7,980,024 and US Pub. No. 2010/0304452. However, the production of ethanol from algae still faces obstacles prior to becoming commercially viable.

One alternative to direct fermentation to ethanol that is proposed is the biochemical and synthetic conversion of biomass to acetic acid, followed by conversion of acetic acid to ethanol using esterification and hydrogenation, as described in U.S. Pat. No. 6,509,180.

Other processes that do not involve fermentation or biochemical conversion have also been proposed. U.S. Pat. No. 7,884,253 discloses the selective production of ethanol from syngas. Syngas derived from cellulosic biomass (or other sources) can be catalytically converted into methanol, which in turn can be catalytically converted into acetic acid or acetates. The acetic acid or acetates can be reduced to ethanol according to several variations. U.S. Pat. No. 7,884,253 claims yields of ethanol from biomass can exceed 100 gallons per dry ton of biomass.

U.S. Pat. No. 4,952,504 discloses a process in which a biomass slurry is hydrolyzed in a fuel fired hydrolysis heater. When the biomass is fruit waste, the organic acid in the waste is used as the hydrolysis catalyst. When the biomass does not contain organic acid, carbon dioxide generated in a fermenter is fed to the hydrolysis heater as carbonic acid to provide the catalyst. Solids are separated from the hydrolyzed effluent, and flue gas from the hydrolysis heater is used to dry the solids. The effluent is fermented and subsequently distilled at substantially atmospheric pressure to produce ethyl alcohol vapor. The vapor is fed to a vacuum distillation tower for producing anhydrous ethanol. A vacuum distillation tower bottoms stream provides the reflux for the atmospheric distillation unit. A portion of the stillage from the atmospheric distillation unit is recycled by mixing with new feedstock. The remaining stillage is evaporated to a syrup, mixed with unreacted solids, and dried to produce an animal feed byproduct.

US Pub. No. 2011/0004034 discloses production of ethanol from a carbonaceous feedstock by first converting the carbonaceous feedstock to synthesis gas, then to ethanoic acid, which is esterified to make ethyl acetate, which is hydrogenated to produce ethanol. The carbonaceous material may include coal, natural gas, and/or biomass.

US Pub. No. 2010/0185021 discloses a method of producing alcohols, hydrocarbons, or both from biomass by converting biomass into a carboxylic acid, reacting the carboxylic acid with an olefin to produce an ester and hydrogenolyzing the ester to produce alcohol. The '021 application also discloses a method of producing hydrocarbons from biomass by converting at least a portion of the biomass into a carboxylic acid, a ketone, or an ammonium carboxylate salt, reacting at least one of a portion of the carboxylic acid, a portion of the ketone, or a portion of the ammonium carboxylate salt in an oligomerization reactor as at least part of a process that produces an oligomerization product and separating hydrocarbons from the oligomerization product.

US Pub. No. 2009/0221725 discloses a process for converting methanol to ethanol which comprises reacting methanol and carbon monoxide in the presence of a catalyst to produce a product comprising at least 25 mole % methyl acetate and, in some instances, acetic acid. The acetic acid then is reacted with at least one alcohol to produce at least one acetate selected from methyl acetate, ethyl acetate, and butyl acetate. The at least one acetate, if produced, and the methyl acetate produced as a result of reacting methanol and carbon monoxide then are hydrogenated to produce ethanol. Syngas may be produced from biomass to produce all or a portion of the methanol, hydrogen, and carbon monoxide requirements for the process.

US Pub. No. 2009/0158663 discloses a method of biomass gasification by combining a biomass feedstock with a light hydrocarbon composition to form a slurry. The method includes the steps of combining a biomass feedstock with water to form a slurry; feeding the slurry to a gasifier to produce a fuel gas; and injecting a light hydrocarbon into the gasifier, to generate gasification temperatures greater than about 900° C., by partial or complete combustion of the light hydrocarbon.

US Pub. No. 2007/0270511 discloses a method and apparatus for gasifying biomass, in a steam gasifier that employs a fluidized bed and heating using hot flue gases from the combustion of synthesis gas. Methods and apparatuses for converting synthesis gas into ethanol are also disclosed using stepwise catalytic reactions to convert the carbon monoxide and hydrogen into ethanol using catalysts including iridium acetate.

US Pub. No. 2006/0141584 discloses a pretreatment of biomass for ethanol production is disclosed. The pretreatment of biomass such as sawdust or wood chips by processing such biomass through an extruder results in a greatly increased recovery of sugar using hydrolysis.

The entire contents and disclosures of each of the above-mentioned patents and applications are hereby incorporate by reference.

In light of the state of the art, the need exists for improved integrated processes for forming syngas from biomass, where the syngas is particularly well suited for forming various industrial chemicals, and in particular ethanol. In addition, the need exists for improved gasification processes that are well suited for adapting to fluctuations in the availability of fossil fuel and biomass feed sources.

### SUMMARY OF THE INVENTION

The present invention relates to integrated processes for producing industrial chemicals, such as alcohols, carboxylic acids, esters, aldehydes, olefins and polymers from biomass. In one embodiment, the invention is to a process comprising the steps of (a) introducing biomass and an oxygen stream, preferably substantially free of nitrogen, to a gasifier, preferably a fluidized bed non-slurry gasifier, and converting the biomass into a product gas, wherein the gasifier is operated at a pressure of at least 10 bar; (b) compressing the product gas at a compression ratio that is less than 5:1, e.g., less than 3:1, to form compressed product gas; (c) directing a first portion of the compressed product gas to an alcohol synthesis reactor to produce methanol; (d) directing a second portion of the compressed product gas to a gas separator to produce a hydrogen stream and a carbon monoxide stream; (e) reacting the carbon monoxide stream with the methanol to produce acetic acid; and (f) reacting the hydrogen stream with acetic acid to produce ethanol and water.

The process optionally further comprises drying the biomass to form dried biomass having less than 20 wt. % water. The process optionally further comprises subjecting the product gas to a water gas shift reaction to increase hydrogen content of the product gas. The ratio of hydrogen to carbon monoxide after subjecting the product gas to the water-gas shift reaction preferably is greater than 1.8:1 or about 2:1. The process optionally further comprises using at least some of the product water in the water gas shift reaction to increase hydrogen content. The fluidized bed preferably is heated to a temperature from 700° C. to 1100° C. The process optionally further comprises reducing carbon dioxide concentration of the compressed product gas to less than 1 mol. %. The process optionally further comprises separating a carbon dioxide stream from the product gas and drying the biomass with the carbon dioxide stream.

The biomass selected may vary widely. In some exemplary embodiments, the biomass comprises a material selected from the group consisting of timber harvesting residues, forest residues, softwood chips, hardwood chips, tree branches, tree stumps, leaves, bark, sawdust, off-spec paper pulp, paper mill wastes, paper mill sludge, highway clippings, wood pellets, pelletized refuse-derived fuel, and combinations thereof. In another embodiment, the biomass comprises a material selected from the group consisting of wheat straw, rice straw, willow, alfalfa, bagasse, and combinations thereof. The biomass may be derived from a second generation biomass source. The biomass is optionally substantially free of sugar. In some aspects, the biomass comprises aquatic biomass, optionally selected from the group consisting of microalgae, macroalgae, microplants, duckweed, water hyacinth, cattails, banana tree stem, kelp, and green algae.

Biomass, as well as biomass-derived syngas, and biomass-derived industrial chemicals, have detectable ¹⁴C isotope contents as compared to fossil fuels such as coal or natural gas. An equilibrium forms in the Earth's atmosphere between constant new formation and constant degradation, and so the proportion of the ¹⁴C nuclei in the carbon in the atmosphere on Earth is constant over long periods. The same distribution ratio n¹⁴C:n¹²C ratio is established in living organisms as is present in the surrounding atmosphere, which stops at death and ¹⁴C decomposes at a half life of about 6000 years. Methanol, acetic acid, methyl acetate, ethyl acetate, ethanol, as well as other derivative chemicals discussed herein, that are formed from biomass-derived syngas would be expected to have a ¹⁴C content that is substantially similar to living organisms. For example, the ¹⁴C:¹²C ratio of the methanol, acetic acid and/or ethanol may be from one half to about 1 of the ¹⁴C:¹²C ratio for living organisms. In contrast, syngas, methanol, acetic acid and/or ethanol or other industrial chemicals derived wholly from fossil fuels, i.e., carbon sources produced over 60,000 years ago, may have no detectable ¹⁴C content.

The hydrogen stream optionally is reacted with acetic acid in the presence of a catalyst comprising a combination of metals selected from the group consisting of platinum/tin, platinum/ruthenium, platinum/rhenium, palladium/ruthenium, palladium/rhenium, cobalt/palladium, cobalt/platinum, cobalt/chromium, cobalt/ruthenium, silver/palladium, copper/palladium, nickel/palladium, gold/palladium, ruthenium/rhenium, and ruthenium/iron.

In one aspect, the hydrogen stream is reacted with acetic acid in the presence of a catalyst comprising: (i) a first metal selected from the group consisting of cobalt, nickel, rhodium, palladium, osmium, iridium, platinum, titanium, zinc, chromium, rhenium, molybdenum, and tungsten; (ii) a second metal selected from the group consisting of molybdenum, tin, chromium, cobalt, vanadium, tungsten, palladium, platinum, lanthanum, cerium, manganese, rhenium, gold, and nickel, provided that the second metal is different than the first metal, wherein the total content of the first and second metals is from 0.1 to 25 wt. %, based on the total weight of the catalyst; (iii) a silicaceous support, and (iv) optionally at least one support modifier selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof.

In another embodiment, the invention is to an integrated process for co-producing acetic acid and ethanol from biomass, comprising: (a) introducing biomass and an oxygen stream to a pressurized gasifier and converting the biomass into a product gas having a hydrogen to carbon monoxide ratio of less than 1.8:1, wherein the product gas is not subjected to a water gas shift reaction; (b) separating a first portion of the product gas into a hydrogen stream and a carbon monoxide stream; (c) directing a second portion of the product gas and a first portion of the hydrogen stream to an alcohol synthesis reactor to produce methanol; (d) reacting the carbon monoxide stream with the methanol to produce acetic acid; and (e) reacting a second portion of the hydrogen stream with less than 90% of the acetic acid to produce ethanol. The molar ratio of acetic acid produced to ethanol produced optionally is from 1:1 to 1:5.

In another embodiment, the invention is to an integrated process for producing ethanol from biomass, comprising: (a) introducing biomass and an oxygen stream to a gasifier and converting the biomass into a product gas, wherein the gasifier is operated at a pressure of at least 10 bar; (b) compressing the product gas at a compression ratio that is less than 3:1 to form a compressed product gas; (c) directing a first portion of the compressed product gas to an alcohol synthesis reactor to produce methanol; (d) reacting carbon monoxide with the methanol to produce acetic acid; (e) reducing a first portion of the acetic acid with hydrogen to produce ethanol in the presence of a catalyst having a selectivity to ethanol that is greater than 50%; (f) esterifying a second portion of the acetic acid and the ethanol to produce ethyl acetate; and (g) reducing the ethyl acetate with hydrogen to produce ethanol, provided that at least 1.5 times more ethanol is produced by reducing the ethyl acetate than is formed by reducing the acetic acid, and wherein none of the ethanol produced by reducing the ethyl acetate is esterified with acetic acid.

In another embodiment, the invention is to an integrated process for producing ethanol from biomass, comprising: (a) introducing biomass and an oxygen stream to a gasifier and converting the biomass into a product gas, wherein the gasifier is operated at a pressure of at least 10 bar; (b) compressing the product gas at a compression ratio that is less than 3:1 to form a compressed product gas; (c) directing a first portion of the compressed product gas to an alcohol synthesis reactor to produce methanol; (d) reacting carbon monoxide with the methanol to produce acetic acid; (e) reducing a first portion of the acetic acid with hydrogen to produce ethanol in the presence of a catalyst having a selectivity to ethanol that is greater than 50%; (f) esterifying a second portion of the acetic acid and the methanol to produce methyl acetate; and (g) reducing the methyl acetate with hydrogen to produce methanol and ethanol, wherein methanol produced by reducing the methyl acetate is recycled to at least one of step (d) or step (f). Optionally, at least 1.5 times more ethanol is produced by reducing the methyl acetate than is formed by reducing the acetic acid.

In another embodiment, the invention is to an integrated process for producing ethanol from biomass, comprising: (a) introducing biomass and an oxygen stream to a gasifier and converting the biomass into a product gas, wherein the gasifier is operated at a pressure of at least 10 bar; (b) compressing the product gas at a compression ratio that is less than 3:1 to form a compressed product gas; (c) directing a first portion of the compressed product gas to an alcohol synthesis reactor to produce methanol; (d) reacting carbon monoxide stream with the methanol to produce acetic acid; (e) reducing the acetic acid with hydrogen to produce ethyl acetate in the presence of a catalyst having a selectivity to ethyl acetate that is greater than 50%; and (f) reducing the ethyl acetate to ethanol.

In another embodiment, the invention is to an integrated process for producing vinyl acetate from biomass, comprising: (a) introducing a biomass and an oxygen stream to a gasifier and converting the biomass into a product gas, wherein the gasifier is operated at a pressure of at least 10 bar; (b) compressing the product gas at a compression ratio that is less than 3:1 to form a compressed product stream; (c) directing a first portion of the compressed product gas to an alcohol synthesis reactor to produce methanol; (d) directing a second portion of the compressed product gas to a gas separator to produce a hydrogen stream and a carbon monoxide stream; (e) reacting the carbon monoxide stream with the methanol to produce acetic acid; (f) reacting the hydrogen stream with at least some of the acetic acid to produce ethanol; (g) dehydrating the ethanol to ethylene; and (h) reacting the ethylene with the remaining portion of the acetic acid to produce vinyl acetate.

In another embodiment, the invention is to an integrated process for producing ethanol from biomass, comprising: introducing biomass and an oxygen stream to a gasifier and converting the biomass into a product gas, wherein the gasifier is operated at a pressure of at least 10 bar; compressing the product gas at a compression ratio that is less than 3:1 to form a compressed product gas; directing a first portion of the compressed product gas to an alcohol synthesis reactor to produce methanol; reacting carbon monoxide with the methanol to produce acetic acid; esterifying the acetic acid with ethanol to form ethyl acetate; and reducing the ethyl acetate with hydrogen in the presence of a catalyst to form ethanol. Preferably, a portion of the ethanol formed in the process is separated and recycled to the esterifying step

In some embodiments, the invention is directed to the co-gasification of aquatic biomass and a fossil fuel. For example, in one embodiment, the invention is to a process for producing syngas, comprising the steps of: (a) introducing aquatic biomass, a fossil fuel (e.g., coal or natural gas), water and oxygen to a gasifier and forming syngas comprising hydrogen, carbon monoxide and carbon dioxide; and (b) feeding aquatic biomass with carbon dioxide derived from the syngas. In this aspect, the gasifier preferably is an entrained flow slagging gasifier. The aquatic biomass that is introduced into the gasifier preferably comprises the aquatic biomass that is fed in step (b). For co-gasification processes, entrained flow, slagging gasifiers are preferred, which preferably operate at pressures greater than 30 bar, optionally greater than 50 bar. Runoff from the gasifier optionally provides nutrients for the aquatic biomass that is fed with carbon dioxide. The aquatic biomass and fossil fuel may be introduced to the gasifier at a weight ratio from 1:99 to 40:60. In preferred embodiments, the aquatic biomass comprises microalgae having an average size of greater than or less than 15 μm.

Optionally, the syngas from the co-gasification process may be used to form various derivative chemicals as described above. For example, The resulting syngas may be compressed, optionally at a compression ratio less than 5:1, e.g., less than 3:1, to form compressed product gas. A first portion of the compressed product gas may be directed to an alcohol synthesis reactor to produce methanol, and a second portion of the compressed product gas may be directed to a gas separator to produce a hydrogen stream and a carbon monoxide stream. The carbon monoxide stream may be reacted with the methanol to produce acetic acid; and the hydrogen stream may be reacted with acetic acid to produce ethanol.

In another embodiment, the invention is to a process for producing syngas, comprising: (a) introducing aquatic biomass, a fossil fuel, water and oxygen to a gasifier and forming a product gas comprising hydrogen and carbon monoxide; (b) increasing the ratio of hydrogen to carbon monoxide using the water gas shift reaction and forming byproduct carbon dioxide; and (c) feeding aquatic biomass with carbon dioxide formed in step (b).

### BRIEF DESCRIPTION OF DRAWINGS

The invention is described in detail below with reference to the appended drawings, wherein like numerals designate similar parts.

FIG. 1 is a flow diagram illustrating an integrated gasification and industrial chemical processes according to an embodiment of the present invention.

FIG. 2 is a flow diagram of a biomass to syngas gasification system.

FIGS. 3A-3F are representations of gasifiers that may be used according to some embodiments of the present invention.

FIG. 4 is an integrated biomass to ethanol production process in accordance with an embodiment of the present invention.

### DETAILED DESCRIPTION OF THE INVENTION

### Introduction

The present invention relates to processes for co-gasifying biomass and a fossil fuel to form syngas, and for forming various industrial chemicals therefrom. In one aspect, for example, the invention is to a process for producing syngas, comprising: introducing aquatic biomass, a fossil fuel, water and oxygen to a gasifier and forming syngas comprising hydrogen, carbon monoxide and carbon dioxide; and feeding aquatic biomass with carbon dioxide derived from the syngas. The syngas may be used, for example, to form methanol, acetic acid, ethanol, methyl acetate or ethyl acetate, among other industrial chemicals.

In another embodiment, the inventive process comprises the steps of: introducing aquatic biomass, a fossil fuel, water and oxygen to a gasifier and forming a product gas comprising hydrogen and carbon monoxide; increasing the ratio of hydrogen to carbon monoxide using the water gas shift reaction and forming byproduct carbon dioxide; and feeding aquatic biomass with carbon dioxide formed in the forming step.

As indicated, in some aspects, the invention further relates to producing industrial chemicals, in particular ethanol and derivatives thereof, from the syngas or product gas. The processes involve specialized gasification techniques for converting biomass to syngas, wherein the syngas is particularly well suited for synthesizing the desired industrial chemical, e.g., alcohol, carboxylic acid, ester, aldehyde, olefin or polymer. The biomass gasification technology preferably operates at a high pressure relative to other biomass gasification technologies. Such gasification processes are generally preferred in that they form pressurized syngas streams having the desired hydrogen and carbon dioxide content at or near the desired pressure for subsequent conversion to the desired product, e.g., methanol, acetic acid or ethanol.

In some embodiments, the invention relates to integrated processes for forming ethanol from biomass via methanol and acetic acid intermediates and in which one or more process streams are recycled within the integrated system. The processes of the invention advantageously allow for the production of bio-chemicals from renewable sources.

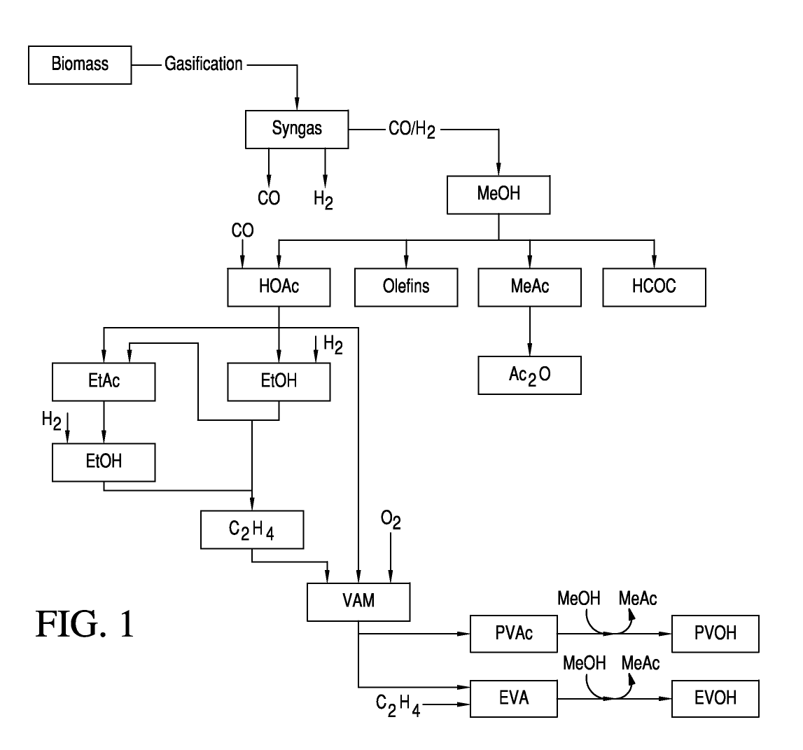

In one embodiment, biomass is gasified to form syngas, which is used in the production of methanol. The methanol that is produced from the biomass may then be used to produce a variety of bio-chemicals including but not limited to a variety of commercially useful chemicals such as olefins, methyl acetate, acetic acid, and formaldehyde. The production of these chemicals may also be integrated with the production of additional derivative products as shown in FIG. 1, and described below. For example, in one embodiment, the gasification process is integrated with a methanol production process and a carbonylation process for forming acetic acid. In this aspect, the process involves converting biomass to syngas, converting the resulting syngas to methanol and converting the resulting methanol to acetic acid. The acetic acid may be reduced with hydrogen derived from the syngas to produce ethanol. Ethanol may be used a fuel or converted to another derivative product such as ethylene, which may be polymerized to form polyethylene. The acetic acid and ethylene, derived either from methanol via conventional methanol to olefin (MTO) technology or from ethanol as described above, may be reacted to produce vinyl acetate (VA). Vinyl acetate may be polymerized to form polyvinyl acetate (PVA). Using a saponification process, the methanol may be used to convert polyvinyl acetate to polyvinyl alcohol (PVOH) Likewise, vinyl acetate may be polymerized with ethylene to form ethylene-vinyl acetate copolymer (EVA or VAE copolymer) and may be converted to ethylene-vinyl alcohol using methanol. As seen, producing syngas from biomass beneficially allows for integration of several chemical processes and production of numerous bio-chemicals.

### Gasification

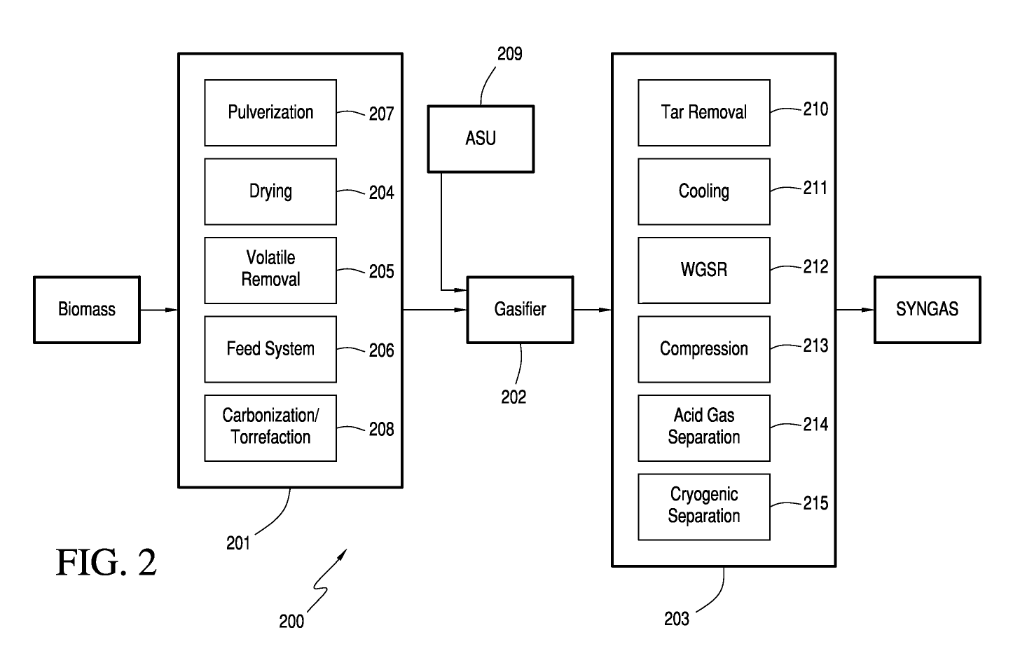

FIG. 2 represents a gasification process 200 that involves biomass pre-treatment 201, gasification 202, and post-treatment 203 of the resulting product gas to form syngas. Various features in the pre-treatment and post-treatment may be combined. In one embodiment, biomass pre-treatment may involve one or more of the following steps: drying 204, removal of volatile components 205, feeding system 206, pulverization 207, and carbonization/torrefaction 208. In addition to biomass pre-treatment, an oxygen generator 209, such as an air separation unit, may be used to form oxygen, which may be used in the gasification step. After pre-treatment, the biomass is fed to gasifier 202 along with the oxygen and heated to an effective temperature to produce a product gas. The product gas is purified in one or more post-treatment steps to form syngas.

Post-treatment 203 may involve one or more of the following steps: tar reforming or removal 210, cooling 211, water-gas-shift-reaction (WGSR) 212, compression 213, acid gas separation 214, and cryogenic separation 215. It should be understood that each of the various steps in pre-treatment and post-treatment may be performed and/or repeated as necessary. In addition there may be further steps added to either the pre-treatment or post-treatment without departing from the scope of the present invention. Each of these steps is described further herein as part of the gasification process 200.

Biomass may be obtained from any suitable source including, but not limited to, agricultural plant waste, plant waste from processes, consumer waste and energy crops grown specifically for fuel production. Examples of biomass include, but are not limited to, agricultural wastes, forest products, grasses, and other cellulosic material, timber harvesting residues, softwood chips, hardwood chips, mixtures of softwood and hardwood chips, tree branches, tree stumps, leaves, bark, sawdust, off-spec paper pulp, corn, corn stover, wheat straw, rice straw, sugarcane bagasse, switchgrass, miscanthus, animal manure, municipal sewer waste (MSW), municipal garbage, municipal sewage sludge, commercial waste, grape pumice, grape seeds, almond shells, pecan shells, coconut shells, coffee grounds, grass pellets, hay pellets, wood pellets, cardboard, paper, cloth, forest residue, paper mill waste, sludge, willow, alfalfa, pelletized refuse derived fuel (RDF), bagasse, highway clippings, and mixtures thereof. In one embodiment, the biomass is obtained from a wood source and may include a material selected from the group consisting of timber harvesting residues, forest residues, softwood chips, hardwood chips, tree branches, tree stumps, leaves, bark, sawdust, off-spec paper pulp, paper mill wastes, paper mill sludge, highway clippings, wood pellets, pelletized refuse-derived fuel, woody biomass crops, and combinations thereof. Wood sources for biomass may be suitable due to low ash content of less than 2%. In some aspects, the biomass comprises aquatic biomass, optionally selected from the group consisting of microalgae, macroalgae, microplants, duckweed, water hyacinth, cattails, banana tree stem, kelp, and green algae. Aquatic biomass may be particularly desirable for co-gasification with fossil fuels, as described below.

In some embodiments, lignocellulosic feedstocks may be used as the source of biomass. Lignocellulosic feedstocks generally include plant biomass such as, but not limited to, grasses, for example, C4 grasses, such as switchgrass, cord grass, rye grass, miscanthus, reed canary grass, or a combination thereof. In general, the biomass may include any suitable type of biomass including, for example, plant biomass, algae biomass, lignocellulosic, cellulosic, and hemicellulosic biomass. In one embodiment, the biomass may comprise at least about 30 wt. % cellulose/hemicelluloses, based on the total weight of the biomass.

Another source of biomass is black liquor, a thick, dark liquid that is a byproduct of the Kraft process for transforming wood into pulp, which is then dried to make paper. Black liquor is an aqueous solution of lignin residues, hemicellulose, and inorganic chemicals. Black liquor may comprise from 15 to 20% solids and contains about half of the original wood chips.

In one embodiment, it is preferred to use a second generation biomass, e.g., lignocellulosic biomass. Preferably, the biomass has low calcium content, low phosphorous content, low potassium content and low silica content. Concentrations of less than 2 wt. %, e.g., less than 1 wt. % or less than 0.5 wt. % each of calcium, phosphorous, potassium, and/or silica are preferred. The biomass optionally has a low sugar content, and optionally is substantially free of sugar. Reduced sugar content beneficially leads to reduced crystallization and reduced “hard candy” formation in the gasifier.

Due to the costs associated with transporting biomass, the biomass source preferably is located relatively close to the gasifier. For example, a majority of the biomass, preferably at least 75 wt. % of the biomass, or at least 85 wt. % of the biomass preferably is obtained from a biomass source located within a 60 mile (97 km) radius of the gasifier, e.g., within a 30 mile (48 km) or within a 20 mile (32 km) radius. These localized radii reduce the energy needed to bring the biomass to the gasifier. In addition, the gasifier may be disposed on a mobile platform, such as a floating vessel or barge, so that the gasifier may be mobile such that it may move to a desired biomass source.

A mobile platform may also be necessary to reach different locations of biomass. Depending on the type of biomass, there may be a harvestable period in which the biomass is available to feed the gasifier. For example, wheat straw, rice straw, willow, alfalfa, bagasse, and combinations thereof, may have a harvestable period. The gasifier is preferably suited to handle multiple types of biomass, including biomass mixtures, to account for the variability of harvestable biomass.

Depending on the source, the biomass may optionally comprise water in an amount from 5 to 55 wt. %, e.g., from 10 to 30 wt. %, the presence of which may reduce gasification efficiency. Prior to being gasified, the biomass may be subjected to pre-processing steps, including but not limited to handling and moisture removal (drying). Moisture removal or drying is especially preferred in the case of high-moisture content biomass. Moisture removal may comprise a separate pre-processing step in a location where waste heat is available or the step may occur in the same location as the gasifier. In one embodiment, carbon dioxide, which is separated from the product gas, may be used to dry the biomass. In another embodiment, nitrogen, e.g., derived from an air separation unit, may be used to dry the biomass. These embodiments reduce the need for relatively expensive natural gas or propane for biomass drying. The heat and energy for the moisture removal step may be derived from an outside source, from burning biomass, or may come from recycled heat and energy formed during the remainder of the biomass process described herein. A pressurized steam dryer may be used to dry the biomass. The biomass, for example, may be heated to a temperature from 200° C. to 400° C. and/or to a pressure from 2 to 4 bar to facilitate moisture removal. After drying, the biomass preferably contains less than 20% moisture, preferably less than 10% moisture. A limited amount of moisture in the feed, however, may facilitate formation of syngas having the desired hydrogen to carbon monoxide ratio, e.g., 2:1 H₂:CO, by in situ water gas shift. Biomass sources may comprise fresh biomass, partially dried biomass, dried biomass or a combination thereof.

Gasification processes may be characterized as either slurry processes or non-slurry processes. In slurry gasification processes, the material to be gasified is pulverized and blended with a carrier medium, typically water, to form a slurry that is subjected to gasification. In preferred embodiments of the invention, the gasification process occurs in a non-slurry gasification process. That is, the biomass preferably is not slurried with a carrier prior to being introduced into the gasifier.

The biomass may be fed via a high-pressure feed system, such as lock hoppers, rotary feeders, plug-forming feeders, and non-plug-forming feeders. The type of feed system may depend on the characteristics of the biomass. To maintain pressure in the gasifier, it is preferred that the feed system operates at a pressure of greater than 10 bar, e.g., greater than 15 bar. In preferred embodiments, the ability of the feed system to transport the biomass into the gasifier, without a slurry, may improve gasifier efficiency and reduce the need for subsequent compression of the product gas. In addition, the feed rate from the reactor should be sufficient to provide a sufficient feed for the scale of the integrated facility. Feed rates may be greater than 5 tons of dry biomass per hour, greater than 25 tons of dry biomass per hour, or from 40 to 60 tons of dry biomass per hour. The lock hoppers are preferably based on gravity discharge of a mostly free-flowing dense, granular and dry biomass material.

Lock hopper systems operate on the principle of intermittent charging or feeding across the pressure boundary, typically by the staged opening and closing of valves on the top and bottom of the charged pressure vessel. Lock hopper systems are non-continuous feeders with long cycle times more suitable to handle dried biomass. In one cycle, the top valve of a lock hopper system may be opened to receive material while the bottom valve is maintained in a closed position. After the top valve is closed, the lock hopper is brought to above system pressure, typically with an inert gas. Following pressurization, the bottom valve is opened, and the material is allowed to discharge to feed the gasifier. This cycle is repeated when the lock hopper is emptied and the vessel is depressurized. In one embodiment, dual or parallel lock hoppers may be employed to allow one lock hopper to be on-line, that is, discharging at pressure to the gasifier, and allow the other lock hopper to be in the filling and pressurizing modes.

Continuous feeders such as rotary feeders or plug-forming feeders may also be used in some embodiments of the present invention. Rotary feeders or plug-forming feeders single-shaft or multi-shaft screw extruders for the charging biomass to the gasifier. Typically, the single-shaft or multi-shaft screw is tapered through the throat. A piston or hopper may be used to compact the biomass into the rotary feeders or plug-forming feeders. After entering the feeder, the screw advances the biomass through the throat causing the densification force to partially dewater the biomass. There may be removable plates in the throat to allow for moisture drainage. Plug-forming feeders may achieve a capacity of greater than 500 tons of dry biomass per day. Lower-density biomass may have capacities that range from 300 to 500 tons of dry biomass per day.

In a preferred embodiment, the gasifier is a fluid bed gasifier that is well suited for processing wood chips or pellets without significant pulverization. The biomass that is introduced into the gasifier may be pelletized or non-pelletized, although it is preferable that the biomass is non-pelletized. Pelletizing may reduce the volume and increase the density of a source of biomass.

Depending on the biomass source employed, pulverization may be possible to increase surface area and improve feedability to the gasifier, although smaller particle sizes may be unsuitable for particularly reactive biomass. The pulverization may involve a size reduction step to reduce the biomass to a particle size of, for example, from 1 micron to 500 microns at its longest dimension, e.g., from 10 to 300 microns, or from 50 to 250 microns. Although smaller particle sizes may result in better reaction rates in thermal processing operations, at some point the energy required to reduce the particle size effectively precludes further size reduction. This is particularly true of particularly fibrous biomass materials, which may be difficult and costly to reduce in particle size. Non-limiting examples of mechanical size reduction equipment include rotary breakers, roll crushers, jet mills, cryogenic mills, hammermills, impactors, tumbling mills, roller mills, shear grinders, and knife mills. When a pulverization technique is used it is preferred to reduce the size prior to moisture and/or volatile removal.

Pretreatment of the biomass may also include carbonization and/or torrefaction steps. Torrefaction is a thermo-chemical treatment in which biomass is heated to temperature from 200 to 350° C., preferably from 200 to 300° C., in absence of oxygen, preferably at atmospheric pressure. Carbonization, while similar to torrefaction, decomposes biomass in the absence of oxygen, but at temperatures greater than 350° C. Both of these pre-treatment processes may remove a significant portion of hydrocarbons, i.e., hemicellulosis, leaving only fixed carbon to be gasified. One advantage of employing fluid bed gasification technology is that pretreatment like torrefaction may not be required, thereby reducing cost.

In addition to biomass, an oxygen stream is also fed to the gasifier. For purposes of the present invention, it is preferred that the oxygen stream comprises at least 50 vol % oxygen, e.g., at least 75 vol. % oxygen or at least 90 vol. % oxygen. The oxygen stream preferably is substantially free of nitrogen. Hence, the oxygen preferably is not delivered to the gasifier in the form of air. In one embodiment, the oxygen stream may contain less than 5 mol. % or less than 1 mol. % nitrogen. Nitrogen may be eliminated from the gasification system by using an air separation unit (ASU) to produce the oxygen stream. ASU's use cryogenic distillation for high-throughput and high-purity oxygen demands. Suitable ASU's provide an oxygen stream having a purity that is greater than 99 mol. % oxygen. In addition, the other gases that are separated, such as nitrogen, may be used as an inert gas in an integrated process. ASU's are described in U.S. Pat. No. 6,425,937 and US Pub. No. 2004/0035150, the entire contents and disclosures of which are hereby incorporated by reference. Although the ASU may require increased capital and operating costs, using an oxygen stream may allow operation of a lower temperature gasifier. Other oxygen supply systems such as pressure swing adsorption (PSA) or polymeric membrane systems may also be used.

Catalytic gasification of biomass to syngas may also be carried out in the presence of steam. For example, biomass can be reformed non-catalytically, optionally in a Klepper gasifier, with superheated steam to produce carbon monoxide and hydrogen. While it is known in the art to use coal-fired boilers to provide the required steam, the use of renewable fuel sources to power the boilers may be desired. In the present invention, the steam may be generated by a boiler powered by a renewable source, such as a wood-fired boiler. The boiler, fueled for example by biomass, can be any of the boilers known to one of ordinary skill in the art. Any water source may be used to generate the steam. However, using purified and/or deoinized water may reduce corrosive processes in the boiler.

The steam produced by the boiler may be fed in whole or in part to within the gasification process or elsewhere in the integrated system. For example, the steam may be fed to the gasifier, cooled syngas, or other areas where steam is desired.

Regardless of the source of biomass, each biomass source generally has similar carbon content, as shown by exemplary gasification formula I.

C:H_{1.4}:O_{0.6}+O₂→CO+0.7H₂ (I)

To produce syngas, pre-processed biomass, oxygen and/or steam are fed to the gasifier. A fluidized bed media, e.g., olivine particles, may be used in the gasifier to facilitate fluidization and to account for the reactive nature of biomass.

Most biomass gasifier technology operates at relatively low pressures, e.g., less than 5 bar, necessitating multiple costly compression stages for many chemical processes that operate at higher pressures. Preferably, the gasification processes employed in the present invention operate pressures of at least 10 bar, e.g., at least 12 bar or at least 15 bar. The pressure of the pressurized gasifier preferably is such that subsequent compression of the product gas or resulting syngas may not be necessary, or if necessary, will be at relatively low compression ratios of on the order of less than 5:1, less than 3:1, less than 2.5:1 or less than 2:1.

Gasifier technology may also be characterized as slagging or non-slagging. Slagging gasifiers typically operate at higher temperatures than non-slagging gassifiers and involve melting ash to form “slag,” a glassy solid that acts to insulate the gasifier. In preferred embodiments of the invention, the biomass gasifier is a non-slagging gasifier, and the gasifiers employed in the invention operate at temperatures that do not form slag. To avoid slag formation, the ash melting point of the biomass should be greater than the operating temperature of the gasifier.

Suitable gasifiers may include a vessel constructed with an exterior of a metal such as carbon steel, with an interior refractory lining sufficient to withstand the temperatures within.

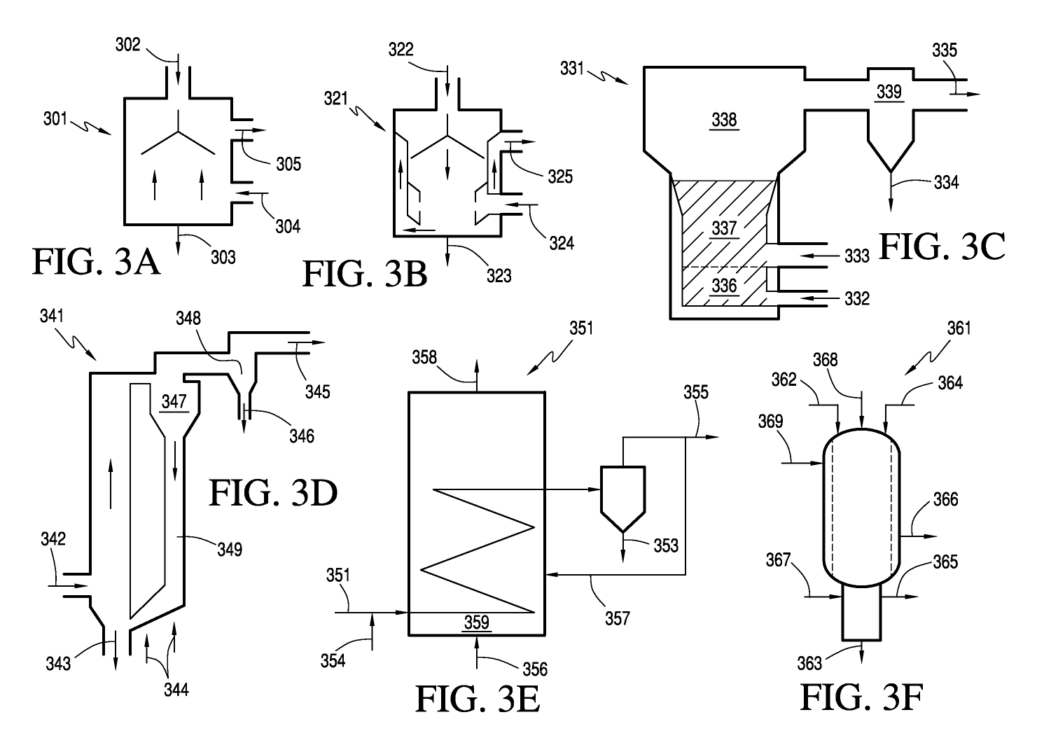

A wide variety of gasifiers may be used with embodiments of the present invention. The gasifier may be a fixed bed gasifier as shown in FIG. 3A-B, a fluidized bed gasifier as shown in FIG. 3C, a circulating fluid bed gasifier as shown in FIG. 3D or an entrained-flow gasifier as shown in FIG. 3E-F.

In a fixed bed gasifier, the configuration may be updraft or downdraft. The biomass is passed through consecutive zones of drying, pyrolysis and char combustion. As shown in FIG. 3A, the biomass enters the top of an updraft gasifier 301 in line 302 via a lock hopper or rotary valve. An oxygen stream enters the gasifier in line 304 and reacts with char in the combustion zone to form carbon monoxide, carbon dioxide and water. The gases flow upward, as shown by the arrows. The biomass flows counterflow to the oxygen stream and goes through the stages of drying, devolatilizing and char combustion. Temperatures in the char combustion zone may be as high as 1200° C. and the hot gas provides energy for the heating, drying and pyrolysis of the biomass feedstock. During pyrolysis, the gas contacts the biomass feedstock at a temperature in the range of 400° C. to 800° C. The biomass is devolatilized to form pyrolysis products and char. Unburned char and ash exit via line 303. The resulting stream, e.g., product gas, exits the gasifier in line 305 at a temperature ranging from 800° C. to 1000° C. Line 305 may also comprise tar and water, which may be subsequently removed. Fixed bed gasifiers are generally less desired for biomass gasification in that they typically operate at lower gas outlet temperatures and at lower average temperatures. As a result, they tend to generate large quantities of tars and oils.

In the gasifier shown in FIG. 3B, biomass enters the top of a downdraft gasifier in line 322 via a lock hopper or rotary valve. An oxygen stream enters the gasifier in line 324 and flows in the same direction as the biomass. An advantage of a downdraft gasifier is that volatiles that were released during the heating of the biomass feedstock enter the char combustion zone where tars are readily cracked. Tar conversion may be greater than 80%, greater than 90% or greater than 99%. The hot char reacts with carbon dioxide and water released during combustion to produce carbon monoxide and hydrogen. Unburned char and ash exit via line 323. The resulting stream, e.g., product gas, exits the gasifier in line 325 at a temperature of approximately 700° C.

Both updraft and downdraft fixed bed gasifiers generally have high ash fusion temperatures to prevent slagging in the combustion zone. Additionally, the moisture content of the biomass feedstock is preferably below 20% to maintain temperatures for tar cracking. Biomass sources that have a low ash content and high ash fusion temperature to prevent slagging are more preferred for fixed bed gasifiers.

In another embodiment, the gasifier may be a fluid bed or bubbling fluid gasifier that operates by passing the oxygen stream upwards through a bed of free-flowing granular materials. The fluid bed gasifier may be operated at a temperature of from 600° C. to 900° C., e.g., from 790° C. to 870° C. Fluidized bed gasification tends to make a much lower amount of by-product tars and oils than fixed bed gasification making it easier to handle the tars and oils in a downstream reformer. Above this range of temperatures, particle agglomeration and bed defluidization may occur. As shown in FIG. 3C, a gas containing steam along with the oxygen stream in line 332 is fed into the plenum 336 of the gasifier. The gas is passed at a large enough velocity for the solid particles to be widely separated and circulated freely throughout the fluid bed 337. The bed material may comprise silica, olivine, limestone, dolomite, alumina, or combinations thereof. When the gas is fed into plenum 336, the superficial velocity of the gas in plenum 336 is controlled to maintain the bed in a fluidized condition. The oxygen stream also serves as an oxidant for the partial oxidation of the biomass feedstock. Biomass is fed into the gasifier via line 333. When line 333 is fed into the gasifier above fluid bed 337, the density of the biomass as compared to the bed material causes the biomass to remain in the top of the gasifier. Preferably, as shown in FIG. 3C, biomass is fed directly into fluid bed 337 via line 333. The biomass in fluid bed 337 is rapidly devolatilized. After passing through the fluid bed, the gas and biomass move to freeboard 338 before passing to cyclone 339. Freeboard 338 has an increased cross sectional area which lowers the superficial gas velocity below fluidization velocity, thus dropping solids out to maintain fluid bed solids in the fluid bed. The increased cross sectional area also allows for the desired gas-phase residence time for complete devolatilization. Cyclone 339 is used to return larger entrained solids to the fluid bed. Ash exits the gasifier in line 334. The product gas exits cyclone 339 in line 335.

In one exemplary embodiment, a bubbling fluid bed gasifier may use parallel lock hoppers to minimized gas usage. The gasifier may be run at a pressure greater than 10 bar, greater than 12 bar or greater than 15 bar, and optionally up to 23 bar. In terms of ranges, the gasifier may be run, for example, at a pressure from 10 to 50 bar, from 12 to 25, or from 15 to 20 bar. In another embodiment, the temperature of the fluidize bed gasifier may range from 700° C. to 1100° C., e.g., 800° C. to 900° C.

In yet another embodiment, the gasifier, as shown in FIG. 3D, may comprise a circulating fluid bed gasifier. Similar to a fluid bed gasifier, biomass feedstock is fed in line 342 and oxygen stream in 344 flows upwards. This type of gasifier has a high solids circulation rate. The turbulent bed solids are collected, separated from the gas, and returned to the bed. The gas and biomass move to first cyclone 347 and then second cyclone 348 for removal of solids. Bottom ash exits through line 343 at the bottom of the gasifier and fly ash exits through line 346 after passing through second cyclone 348. The product gas exits the gasifier through line 345.

Entrained-flow gasifiers, as shown in FIG. 3E-F, are well known in the art for the purpose of gasifying coal or a combination of biomass and coal. Due to the slurry fed and use of non-renewable sources, the entrained-flow gasifier may be less preferred with embodiments of the present invention. Such gasifiers are typically run at high temperatures, particularly from 1300° C. to 1400° C., so that the gasification temperature is higher than the melting point of coal ash. This results in a liquid slag which is then removed from the gasifier. The biomass may be torrefied, a mild form of pyrolysis carried out at temperatures ranging from 200° C. to 320° C. The torrefied biomass is pulverized into small particles with a particle size of 100 microns to 1 millimeter, and mixed with a liquid, such as water, to form a torrefied slurry feed. The torry slurry feed is then fed to the gasifier, either in dry or liquid form. In FIG. 3E, dry biomass slurry in line 351 is combined with steam in line 354 and fed to furnace 359. The oxygen stream is introduced into furnace 359 via line 356. Flue gas exits the gasified via line 358 while char exits via line 353. Product gas exits the gasifier via line 355 and recycle gas is sent back to the gasifier in line 357. In FIG. 3F, a biomass slurry is fed to the gasifier in line 362, along with the oxygen stream in line 364, water in line 369 and quench water in line 367. Natural gas may be fed to the gasifier in line 368. Slag exits the gasifier in line 363. Steam exits the gasifier in line 366 and syngas exits the gasifier in line 365.

In an exemplary embodiment, an entrained-flow gasifier may be modified to conform to biomass instead of coal. This gasifier uses a low-temperature gasification stage to dry the biomass to a moisture content of from 10 to 30%, e.g., from 15 to 20%. The biomass may be fed to the gasifier via lock hoppers. The biomass is then carbonized through low-temperature pyrolysis to form pyrolysis gas. The pyrolysis is typically conducted with an external heating medium, e.g., indirect steam heat, at a temperature from 400° C. to 500° C. During this low-temperature oxidation, the biomass is separated into char and pyrolysis gas containing a high tar content. The second stage is a high-temperature gasification process in which the pyrolysis gas is partially oxidized at a temperature of above 400° C. This exothermic oxidation results in temperatures which break down tar in the pyrolysis gas. The final stage uses the char resulting from the first stage in an endothermic gasification step. The char is combined with the hot combustion products from the second stage and the carbon from the char reacts with carbon dioxide to generate additional carbon monoxide and hydrogen in the syngas product. This is an endothermic reaction and reduces the syngas product temperature to approximately 900° C.

In one embodiment, biomass, in particular aquatic biomass, and a fossil fuel, optionally coal or natural gas, may be co-gasified in a gasifier, preferably an entrained flow slagging gasifier, to produce syngas comprising hydrogen, carbon monoxide and carbon dioxide. The carbon dioxide formed in this process may advantageously be used to feed the biomass prior to being harvested for gasification, thereby exhibiting a synergy with respect to overall emissions, including greenhouse gas emissions, without sacrificing the energy content of the product gas. For example, the carbon dioxide may be separated and bubbled through spargers into the water in which the biomass is growing. Carbon dioxide separation may be by any convenient technique, such as with one or more amines (e.g., monoethanol amine system), membranes, cryogenic techniques, or molecular sieves. By using the carbon dioxide to grow biomass, overall carbon dioxide production can be advantageously reduced. In addition, in co-gasification processes, adjusting the amount of fossil fuel, e.g., coal, fed to the gasifier can advantageously alleviate biomass feedstock fluctuations. This co-gasification approach may also allow biomass feedstocks to benefit from the same economies of scale as achieved with coal gasification. In addition, by employing biomass in the gasifier feed, cleaner syngas may be advantageously formed having lower sulfur and ash content and having a neutral carbon footprint.

As an additional benefit, runoff from the gasifier may provide nutrients, e.g., nitrogen, phosphorus, potassium, for the biomass prior to harvesting. In this manner, a desirable syngas composition, preferably suitable for forming methanol, may be formed while advantageously increasing biomass production. If desired, the ratio of hydrogen to carbon monoxide may be altered using the water gas shift reaction. For example, if the hydrogen to carbon monoxide ratio is too low, it may be increased using the water gas shift reaction, optionally forming byproduct carbon dioxide for feeding the biomass. In this manner, the low hydrogen content of syngas formed from coal may be advantageously increased to make syngas suitable for methanol production while simultaneously increasing carbon dioxide content, which may be separated and advantageously used for biomass growth.

In this aspect, the aquatic biomass is preferably selected from the group consisting of microalgae, macroalgae, microplants, duckweed, water hyacinth, cattails, banana tree stem, kelp, and green algae. The aquatic biomass optionally may be formed from microalgae having an average size less than 15 μm or from macroalgae having an average size greater than 15 μm. For co-gasification processes, entrained flow, slagging gasifiers are preferred, which preferably operate at pressures greater than 30 bar, optionally greater than 50 bar, or from 30 to 60 bar. The amount of aquatic biomass relative to the amount of fossil fuel introduced to the gasifier in this embodiment may vary widely, but optionally is from 1:99 to 40:60, from 10:90 to 30:70 or about 20:80.

In another embodiment, instead of the direct addition of the oxygen stream to the gasifier, a gasifier may be indirectly heated. In an indirect gasifier, pipes or heat exchangers are heated within the reactor. A heating medium is passed inside of the pipes, which maintains the reactor at the desired temperature. Steam is typically used as the heating medium. The gasifier is typically operated at a temperature of from 600° C. to 700° C.

From each of the various gasifiers a product gas is produced that comprises carbon monoxide, hydrogen, and carbon dioxide. The product gas may also comprise methane, tars, and other volatile matter. Because an oxygen stream is used, the product gas preferably does not contain nitrogen in any appreciable amount. For example, the product gas may comprise less than 1 vol % nitrogen, less than 0.5 vol % nitrogen or less than 0.01 vol % nitrogen. To further recover syngas suitable for integration with one or more industrial chemicals, the product gas may be further processed.

Depending on the gasification process, it may be necessary to remove tar and volatile matter. Tars may be reduced by using a catalyst in the gasification process such as dolomite. Tars and phenolic hydrocarbons produced in a gasifier may require removal to reduce emissions, avoid fouling, and avoid poisoning catalysts in the integrated process. As compared to coal, biomass has a larger amount of total volatile matter, such as phenolics. During gasification, the volatile matter is released at relatively low temperatures. A large part of this volatile material is responsible for the tar formation during gasification of biomass. Tar levels in the syngas also depend largely on the gasifier configuration. In an updraft fixed bed gasifier operated between 300° C. to 1000° C., the product gas contain may contain up to 35000 ppm tar, when not controlled. Tars have relatively high heat contents and can be burned in combustors, but they may plug char filters and may form soot during combustion. Therefore, to maximize system efficiency, the tar may optionally be removed from the gasified biomass.

Tar may be removed from the gasified biomass product through a catalytic tar reformer or thermal tar cracker. The catalytic tar reformer may be operated at a temperature comparable to the gasifier temperature, preferably below 900° C. However, the temperature should not drop below the tar dew point of about 540° C.

After gasification and optional tar removal, the product gas will be partially cooled to minimize the amount of alkali vapors. The cooling may occur at a temperature from 350° C. to 650° C. Optionally, the product may then pass through a filter to remove solids. A downstream of steam may be purged from the system or recycled to the gasifier.

Depending on the H₂ to CO ratio, in some embodiments, steam may be added to the gasifier to promote the water gas shift reaction (WGSR) to increase the hydrogen concentration of the syngas. For many industrial chemical production processes, such as the production of methanol, generally a ratio of about 2:1 is preferred. Biomass generally comprises more carbon than hydrogen, relative to natural gas or coal, and thus the ratio of H₂ to CO in the product gas is typically less than 2:1, e.g., less than 1.8:1, less than 1.6:1 or less than 1.4:1. The WGSR may be used during any point in the gasification process. In some embodiments, there may be multiple WGSR. After the WGSR the product gas may comprise a H₂ to CO ratio that is greater than 1.8:1, e.g., greater than 1.9:1. Preferably, the H₂ to CO ratio is from 1.8:1 to 4:1, e.g., from 1.9:1 to 3:1.

When biomass gasification is integrated with ethanol production, there may be some water that is co-produced with the ethanol. The water stream may be separately recovered and used in the WGSR to increase the hydrogen content. Various ethanol/water separation processes are described in co-pending U.S. patent application Ser. Nos. 13/094,537; 13/094,657; 13/094,473; and 13/094,588, the entireties of which are incorporated herein by reference.

In some embodiments, the WGSR may not be necessary depending on the desired bio-chemical products to be produced. Syngas having a H₂:CO ratio of about 2:1 is desired for the production of methanol. A portion of this syngas having a H₂:CO ratio of about 2:1 may be separated into a carbon monoxide stream and a hydrogen stream. The separated carbon monoxide stream may be used to carbonylate the methanol to form acetic acid, and the separated hydrogen stream may be used to hydrogenate the acetic acid to form ethanol.

Depending on commercial demands, it may be desirable to form a syngas stream having a H₂:CO ratio of less than 2:1 to coproduce acetic acid and ethanol. For example, syngas having a H₂:CO ratio of less than 2:1, i.e., less than the stoichiometric amount necessary to form and hydrogenate acetic acid, may be separated as described above. The separated carbon monoxide stream may be used to form acetic acid and the separated hydrogen stream may be used to hydrogenate some of the acetic acid. Since less hydrogen is available due to the low H₂:CO ratio, the acetic acid is only partially hydrogenated resulting in the coproduction of acetic acid and ethanol.

In addition to WGSR, syngas having a high H₂:CO ratio may be achieved through steam reforming of methane prior to being integrated with an industrial chemical production process.

In one embodiment, the product gas may be further compressed after exiting the gasifier. As indicated above, it is preferred to operate the gasifier at a high pressure. The product gas preferably is compressed to a pressure of at least 25 bar, e.g., at least 35 or at least 40 bar. Preferably, the product gas is compressed at a compression ratio that is less than 3:1, e.g., less than 2.5:1 or less than 2:1. The temperature of the compressor may be from 220° C. to 300° C. A heat supplier may be used to heat the gas to the operating temperature. The compressor may be a four-stage reciprocating compressor with interstage cooling on all stages. Each stage may be equipped with a water separator to remove the water condensed in each state.

Preferably, the product gas is compressed during the gasification so that the syngas or products of the syngas, e.g., hydrogen and carbon monoxide, do not require further compression or require minimal compression when being integrated with the various chemical processes.

Following compression, the carbon dioxide content of the product gas may be reduced. Upon exiting the gasifier, the product gas may have a carbon dioxide concentration of greater than 1 mol. %, or greater than 5 mol. %, and more typically from 10 to 40 mol. %, e.g., from 15 to 30 mol. %. In one embodiment, the product gas may be separated into a carbon dioxide stream and a syngas stream. To accomplish this, the product gas may be fed to an acid gas removal device that uses a solvent to remove acid gas, e.g., carbon dioxide. Solvents may include methanol, dimethyl ether of polyethylene glycol (DEPG), N-methyl-2-pyrrolidone (NMP), N-methyl-diethanolamine (MDEA), and propylene carbonate. A suitable acid gas removal process is the RECTISOL™ process developed by Lurgi. This process uses methanol as a solvent to remove sulfur and sulfur containing compounds such as hydrogen sulfide gas. The process also removes carbon dioxide. The methanol solvent may removal contaminants from the syngas, including ammonia, mercury and hydrogen cyanide.

The purged carbon dioxide may be sequestered. In one embodiment, the purged carbon dioxide may be used to dry the biomass in the pre-processing steps. In another embodiment the carbon dioxide may be used in an enhanced oil recovery process.

Other methods of removing carbon dioxide may include chemical solvents, physical solvents, membranes, and cryogenic fractionation. A membrane may be used when the product gas is at a higher pressure and contains large concentrations of carbon dioxide.

After carbon dioxide and sulfur removal, the syngas from the gasification of biomass may be used to produce one or more industrial chemicals. Any product gas that is not converted to syngas during the post-treatment may be used a low grade fuel source. Depending on the integration, it may be necessary to split a portion of the syngas into a hydrogen stream and a carbon monoxide stream.

In one embodiment, after acid gas removal, the product gas stream may be separated into two or more streams. At least a portion of the stream comprising carbon monoxide and hydrogen preferably is fed to a methanol production unit while at least a second portion of the stream, also comprising carbon monoxide and hydrogen, may be fed to a cryogenic separator, operating for example at a temperature of −170° C. to −200° C., for further processing and purification. The cryogenic separator may comprise one or more of a wash column, a flash column, and/or a distillation column. In some embodiments, the cryogenic separator may further remove trace impurities of gas, such as methane and argon. At least one hydrogen rich stream preferably is withdrawn from the cryogenic separator and fed to an integrated chemical production process, such as a hydrogenation process to produce ethanol. At least one carbon monoxide rich stream is withdrawn from the cryogenic separator and fed to the acetic acid unit described below.

In some embodiments, a hydrogen permeable polymeric membrane may be used to separate a portion of the syngas into a hydrogen stream and carbon monoxide stream. Alternatively, a pressure swing adsorption (PSA) or cryogenic purification technique may be used separate a portion of the syngas.

Regardless of the gas separation method, it is preferred that the hydrogen stream and carbon monoxide stream are relatively pure, e.g., each having a purity greater than 97 mol. %., e.g., greater than 99 mol. %. Operating the gasifier with an oxygen stream as opposed to air results in syngas that may contain less nitrogen and allows for improved recovery of a pure carbon monoxide stream.

Depending on how the gasification and chemical production processes are integrated, various combinations of the biomass pre-treatment process, gasifiers, or post-treatment of the product gas to syngas may be used. In one embodiment, the gasification process may include a dryer for the biomass, a feed system, a fluidized bed gasifier, tar removal, cooling, WGSR, and acid gas separation.

### Methanol Unit

As shown in FIG. 1, the syngas formed in the gasification step preferably is converted to methanol by reacting hydrogen with carbon monoxide in the presence of a methanol synthesis catalyst. To enhance efficiencies, the syngas produced in the biomass gasification step preferably is at an elevated pressure, as described above, of at least 10 bar, e.g., at least 12 bar, at least 15 bar or at least 20 bar. In preferred embodiments, the methanol synthesis reaction occurs at a pressure of at least 30 bar, at least 35 bar or at least 40 bar. Thus, depending on the pressure of the syngas after the gasification step, it may be necessary to further compress the syngas to the desired pressure for methanol synthesis. Preferably, however, the syngas is compress in fewer than 2 stages, and preferably at a compression ratio less than 3:1, e.g., less than 2:1. Although lower pressures may be used in the gasification step, the energy requirements necessary to subsequently compress the syngas to desired pressures for methanol synthesis renders such low pressure gasification processes generally undesirable for integrated processes.

Two molecules of hydrogen must react with one molecule of carbon monoxide to form one molecule of methanol with water as a byproduct. Methanol is typically produced from the catalytic reaction of syngas in a methanol synthesis reactor in the presence of a heterogeneous catalyst. The catalyst may be a copper-based catalyst, which additionally includes an oxide of at least one element selected from the group consisting of silver, zinc, boron, magnesium, aluminum, vanadium, chromium, manganese, gallium, palladium, osmium, and zirconium. Preferably, the catalyst contains copper oxide and an oxide of at least one element selected from the group consisting of zinc, magnesium, aluminum, chromium, and zirconium. More preferably, the catalyst contains oxides of either or both copper and zinc. For example, in one synthesis process, methanol is produced using a copper/zinc catalyst in a water-cooled tubular methanol reactor. Additional methanol synthesis catalysts include Cu/ZnO/Al₂O₃ and Cu/ZnO/Cr₂O₃. In a gas phase process, the product gas reacts on the surface of the catalysts in a reactor, preferably a fixed bed reactor.

While the byproduct water can be distilled from the product methanol, such a distillation process requires the addition of significant energy and equipment. It would be desirable to save the energy and equipment cost by eliminating the separation of water and methanol. The elimination of the water/methanol separation is enabled if the crude methanol produced has an inherently low water content. Crude methanol with low water content may be produced if the amount of CO₂ in the syngas feedstock is minimized. As previously described, the CO₂ content is minimized by the acid gas removal step. As a result, in one embodiment, the syngas used in the methanol synthesis process has a low CO₂ content resulting in reduced water formation.

Examples of methanol synthesis processes include batch processes and continuous processes. Continuous processes are preferred. Tubular bed processes and fluidized bed processes are particularly preferred types of continuous methanol production processes. The methanol synthesis process is effective over a wide range of temperatures. In one embodiment, the synthesis gas is contacted with the methanol synthesis catalyst at a temperature in the range from 150° C. to 450° C., preferably in a range from 175° C. to 350° C., more preferably in a range from 200° C. to 300° C. The process is also operable over a wide range of pressures. In one embodiment, the synthesis gas is contacted with the methanol synthesis catalyst at a pressure in the range from 1.5 MPa to 12.5 MPa, preferably in a range from 2.0 MPa to 10.0 MPa, more preferably in a range from 2.5 MPa to 7.5 MPa.

Gas hourly space velocities can vary as desired. Preferably, the gas hourly space velocity of flow of gas through the catalyst bed is in the range from 50 hr⁻¹ to 50,000 hr⁻¹. Preferably, the gas hourly space velocity of flow of gas through the catalyst bed is in the range from 250 hr⁻¹ to 25,000 hr⁻¹, more preferably from 500 hr⁻¹ to 10,000 hr⁻¹.

### Methanol to Olefins

Olefins have been traditionally produced from petroleum feedstock by catalytic or steam cracking processes. These cracking processes, especially steam cracking, produce light olefins such as ethylene and propylene from a variety of hydrocarbon feedstocks. As the demand for petroleum increases, new sources of starting materials to produce olefins, especially renewable sources, are needed. Ethylene and propylene are important commodity petrochemicals useful in a variety of processes for making plastics and other chemical compounds. Ethylene is used to make various polyethylene plastics, and in making other chemicals such as vinyl chloride, ethylene oxide, ethylbenzene and alcohol. Propylene is used to make various polypropylene plastics, and in making other chemicals such as acrylonitrile and propylene oxide.

The petrochemical industry has known for some time that oxygenates, especially alcohols, are convertible into light olefins. For example, methanol may be converted to ethylene and/or propylene in the presence of a molecular sieve catalyst. This process is referred to as the methanol-to-olefin (MTO) reaction process. Bio-olefins from the syngas produced according to embodiments of the present invention may be obtained by converting biomass-derived methanol to ethylene and/or propylene in an integrated process. In particular, the integrated system may involve the production of ethylene and propylene from methanol obtained by converting syngas from biomass.

MTO reaction systems are widely known, and described, for example, in U.S. Pat. No. 5,714,662, the entirety of which is incorporated herein by reference. MTO reaction systems involve a process for the production of light olefins from a hydrocarbon gas stream by a combination of reforming, oxygenate production, and oxygenate conversion. U.S. Pat. Nos. 4,328,384 and 4,547,616 teach conversion of oxygenates to olefins using a fluidized zeolite catalyst. Suitable catalysts may also include silicoaluminophosphate molecular sieves, such as SAPO-34, or ZSM-5 type catalysts supplied from Sud-Chemie. A typical MTO reaction system may also include an oxygenate/MTO byproduct separation system, which is adapted to separate unreacted oxygenates from MTO byproducts such as water.

### Methanol to Methyl Acetate

In one embodiment, biomass-derived methanol may be further integrated with a methyl acetate production facility. Methyl acetate is widely used in the production of acetic anhydride, acetic acid, and ethyl acetate, as well as in diluents, coatings, ink resins, and/or solvents in glues, paints, and nail polish removers. Various methods have been used in the past for the production of the methyl acetate, including esterification or direct synthesis.

Methyl acetate can be produced, for example, by the esterification of methanol with acetic acid. An exemplary methyl acetate esterification process is described in U.S. Pat. No. 5,296,630, the disclosure of which is incorporated by reference in its entirety. Acetic acid may also be formed in the carbonylation of methanol as described below. As a results, both of the reactants, methanol and acetic acid, that used to form methyl acetate may be advantageously derived from biomass. The process parameters for the esterification step may vary widely depending, for example, on the catalyst employed and the ester being formed. In one embodiment, an esterification unit operates at a base temperature of from 100° C. to 150° C., e.g., from 100° C. to 130° C., or from 100° C. to 120° C. In terms of pressure, the esterification unit may be operated at atmospheric pressure, subatmospheric pressure, or superatmospheric pressure. For example, in some embodiments, the esterification unit operates at a pressure of from 50 kPa to 500 kPa, e.g., from 50 kPa to 400 kPa, or from 50 kPa to 200 kPa.

The esterification of methanol and acetic acid to form methyl acetate may be performed in the presence of an acidic catalyst. Non-limiting examples of acidic catalysts include macroporous strong-acid cation exchange resins such as those from the Amberlyst™ series distributed by Rohm and Haas. Additional ion exchange resins suitable for use in the present invention are disclosed in U.S. Pat. Nos. 4,615,806, 5,139,981, and 7,588,690, the disclosures of which are incorporated by reference in their entireties. In other embodiments, the esterification unit comprises an acid selected from the group consisting of sulfuric acid, phosphoric acid, sulfonic acids, heteropolyacids, other mineral acids and a combination thereof. In other embodiments, acid catalysts include zeolites and supports treated with mineral acids and heteropolyacids.

In another embodiment, methyl acetate may be produced by reacting methanol and carbon monoxide, as described in U.S. Pat. No. 4,484,002, the disclosure of which is incorporated by reference in its entirety. A mixture of methanol and carbon monoxide with a catalyst composition is heated at an elevated temperature and pressure for sufficient time to produce the desired methyl acetate, and then the methyl acetate is recovered from the reaction mixture. The catalyst composition comprises a ruthenium-containing compound, such as ruthenium oxide, a cobalt-containing compound, such as dicobalt octacarbonyl, and a quaternary onium base or salt, such as tetra-n-butylphosphonium bromide. The catalyst composition is highly selective for the formation of methyl acetate with high conversion rates for the methanol. This embodiment also avoids the use of iodine and other promoters which are difficult to utilize in large scale commercial operations. The process is characterized by the high selectivity in the conversion of the methanol to methyl acetate. Typical conversion of methanol ranges from 70% to about 100%, with selectivities of methyl acetate ranging from 60% to 90%. Valuable byproducts of the reaction include ethyl acetate and acetic acid.

### Methyl Acetate to Acetic Anhydride

In another integrated process, the methyl acetate produced may be further carbonylated to produce acetic anhydride (Ac₂O). Acetic anhydride may also be obtained from carbonylating dimethyl ether or mixtures of dimethyl ester and methyl acetate. An exemplary acetic anhydride production process is described in U.S. Pat. No. 4,374,070, the disclosure of which is incorporated by reference in its entirety. Acetic anhydride has many uses such as in the manufacture of cellulose acetate from cellulose. Acetic anhydride generally is prepared by the carbonylation of methyl acetate (MeOAc) in the presence of a homogeneous Group VIII catalyst, such as rhodium. An iodide promoter, such as lithium iodide (LiI), may also be used. The acetic anhydride carbonylation is typically carried out under anhydrous conditions. The carbon monoxide needed for the reaction may be separated from the syngas produced by the biomass gasification. Heterogeneous catalysts may also be used in some embodiments, such as those described in U.S. Pat. No. 4,328,125, the disclosure of which is incorporated by reference in its entirety.

In some integrated processes, acetic anhydride and acetic acid may be co-produced by carbonylating methyl acetate, as described in U.S. Pat. No. 7,906,680, the disclosure of which is incorporated by reference in its entirety.

### Methanol to Formaldehyde

Methanol produced from biomass-derived syngas may be further processed to form formaldehyde in another embodiment. Some processes for making formaldehyde employ catalysts comprising the oxides of a large number of catalytic elements. Typically such processes are conducted with an excess of oxygen and are characterized by very high conversions even in a single reaction stage. Other processes employ two sequential reaction stages with the total amount of molecular oxygen being less than that required stoichiometrically to convert the methanol to formaldehyde, hydrogen, and water. Various catalysts are known for processes of this type, including silver, used either on an inert support or as the metal itself in the form of gauze or crystals. The catalyst may also be in the form of metallic foam. Process parameters such as space velocity in the catalyst beds are important in controlling the system to optimize process throughput and reaction efficiency. It is also known to introduce additional methanol, molecular oxygen, or both in between the two reaction stages although references to using supplemental methanol between stages where both stages use silver have not been seen.

U.S. Pat. No. 3,959,383 discloses a two-stage reaction system in which methanol is reacted with molecular oxygen to produce formaldehyde. The first stage is carried out in the presence of a silver gauze catalyst although other forms of silver are not excluded, while the second stage employs 20-30 mesh electrolytically prepared silver crystals. Supplemental air is introduced between the two reaction stages, but supplemental methanol is not. The use of silver crystals as a methanol-oxidation catalyst is discussed in U.S. Pat. Nos. 1,968,552 and 1,937,381, the entireties of which are incorporated by reference. The manufacture of electrolytic silver crystals suitable for use as catalysts in those processes has been generally known in the art. U.S. Pat. No. 2,462,413 discloses a two-stage reaction using a supported silver catalyst in each stage. Inter-stage cooling is employed, and additional reaction air is added between the two stages. U.S. Pat. No. 2,519,788 discloses a two-stage reaction in which the first-stage catalyst is metallic silver while the catalyst in the second stage is of the metal oxide type.

U.S. Pat. No. 2,908,715 discloses a single-stage oxidation over a silver catalyst, with space velocity being discussed as a factor which can be manipulated to cope with the problem of localized overheating of the catalyst. U.S. Pat. No. 2,504,402 discloses a multistage catalytic oxidation of methanol. Reactants are cooled between stages, and, according to the patentee, any “well-known catalyst” can be employed, although oxide catalysts are exemplified.

### Methanol to Gasoline (MTG)

In another embodiment, methanol produced by biomass-derived syngas may be further processed to directly make gasoline through well-known methanol to gasoline (MTG) technology. This process typically involves dehydrating methanol to form dimethyl ether (DME), which is then further dehydrated over a zeolite catalyst, such as ZSM-5, to form gasoline with over 80 wt. % C₅₊ hydrocarbons, based on total organics in the product stream. ZSM-5 and other MTG catalysts are selective for gasoline range molecules (C₄-C₁₀), and have a very low selectivity for hydrocarbons above C₁₀. MTG processes are described, for example, in U.S. Pat. Nos. 4,035,430; 4,348,486; 4,404,414; 4,788,042; 4,788,369; and 5,602,289, the entireties of which are incorporated herein by reference.

In one aspect, a TIGAS gasoline production process may be employed. In the TIGAS process, syngas is converted into gasoline in a single-loop process that eliminates the requirement for upstream methanol production and intermediate storage. The process integrates methanol/DME synthesis and subsequent conversion into gasoline in a single synthesis loop. The primary difference between typical MTG processes and TIGAS processes is that the MTG process separately synthesizes methanol, whereas the TIGAS process allows the methanol to react to form DME immediately. The TIGAS process desirably allows for a reduction in reaction pressure and unconverted syngas recycle. See, e.g., Rostrup-Nielsen et al., Polygeneration—Integration of Gasoline Synthesis and IGCC Power Production Using Topsoe's TIGAS Process, Risø-R-1608(EN) 56-68; J. Topp-Jørgensen, “Topsøe Integrated Gasoline Synthesis—The TIGAS Process,” Methane Conversion, 1988; J. Topp-Jørgensen, J. R. Rostrup-Nielsen, “Integrated Process Offers Lower Gas-to-Gasoline Investment,” Oil & Gas Journal, May 19, 1986; F. Joensen et al., “Conversion of Synthesis Gas to High Octane Gasoline,” Proc. 6th Int. Symp. On Large Chemical Plants, Antwerp, Belgium 1985; and U.S. Pat. Nos. 4,481,305; 4,536,485; 7,078,578; 7,517,374; 7,820,867 and 8,067,474, the entireties of which are incorporated herein by reference.

### Methanol to Acetic Acid

In one embodiment, methanol produced by biomass-derived syngas may be further processed to make acetic acid. In a preferred embodiment, the methanol is reacted with carbon monoxide from a carbon monoxide rich stream in an acetic acid synthesis unit to produce acetic acid. Preferably, the carbon monoxide rich stream is obtained by separating a portion of the biomass-derived syngas that is not reacted to form methanol. As described above, the carbon monoxide rich stream may be withdrawn from the cryogenic separator and fed to the acetic acid unit. In some embodiments, the carbon monoxide rich stream may also be fed from an alternative source.

For biomass-derived syngas having a low H₂:CO ratio, e.g., less than 2:1, less than 1.8:1, or less than 1.6:1, the syngas may be separated, e.g., through cryogenic separation unit, into a CO rich stream and a hydrogen rich stream. A portion of the syngas enriched with carbon monoxide may be directed to the acetic acid synthesis unit and the remaining hydrogen rich portion of the syngas, preferably having a H₂:CO ratio greater than 1.8:1, from 1.8:1 to 2.2:1 or about 2:1, may be used to produce methanol and/or in the synthesis of another chemical compound, e.g., in a hydrogenation process such as the hydrogenation of acetic acid to make ethanol, the hydrogenolysis of ethyl acetate to make ethanol, or the hydrogenolysis of methyl acetate to make methanol and ethanol. In this manner, a biomass-derived syngas stream may be advantageously integrated into multiple chemical production processes. Additionally, the syngas may be used in heat recovery to generate steam that may be used throughout the facility, e.g., to heat one or more reboilers in a downstream separation scheme.

The acetic acid synthesis unit may be any of those well known and commercially available to those skilled in the art to form acetic acid from carbon monoxide and methanol. Among currently employed processes for synthesizing acetic acid, one of the most useful commercially is the catalyzed carbonylation of methanol with carbon monoxide as taught in U.S. Pat. No. 3,769,329, incorporated herein by reference. The carbonylation catalyst may comprise rhodium or iridium, either dissolved or otherwise dispersed in a liquid reaction medium or supported on an inert solid, along with a halogen containing catalyst promoter as exemplified by methyl iodide. The patentees disclose a very large number of suitable promoters, most of which are organic iodides. Most typically and usefully, the reaction is conducted by continuously bubbling carbon monoxide gas through a liquid reaction medium in which the catalyst is dissolved.

An improvement in the prior art process for the carbonylation of an alcohol to produce the carboxylic acid having one carbon atom more than the alcohol in the presence of a rhodium catalyst is disclosed in commonly assigned U.S. Pat. Nos. 5,001,259; 5,026,908; and 5,144,068; and EP 0161874 B2, each of which is incorporated herein by reference in its entirety. As disclosed therein, acetic acid is produced from methanol in a reaction medium containing methyl acetate, methyl halide, especially methyl iodide, and rhodium present in a catalytically effective concentration. These patents disclose that catalyst stability and the productivity of the carbonylation reactor can be maintained at high levels, even at very low water concentrations, i.e., 4 weight percent or less, in the reaction medium (despite the general industrial practice of maintaining approximately 14-15 wt. % water) by maintaining in the reaction medium, along with a catalytically effective amount of rhodium and at least a finite concentration of water, a specified concentration of iodide ions over and above the iodide ion that is present from hydrogen iodide. This iodide ion is from a simple salt, with lithium iodide being preferred. The patents teach that the concentration of methyl acetate and iodide salts are significant parameters in affecting the rate of carbonylation of methanol to produce acetic acid, especially at low reactor water concentrations. By using relatively high concentrations of the methyl acetate and iodide salt, one obtains a surprising degree of catalyst stability and reactor productivity even when the liquid reaction medium contains water in concentrations as low as about 0.1 wt %, so low that it can broadly be defined simply as “a finite concentration” of water. In terms of ranges, the water may be present in the reaction medium, for example, in an amount from 0.1 to 10 wt. %, from 0.1 to 5 wt. %, from 0.1 to 2 wt. % or from 0.1 to 1 wt. %. Furthermore, the reaction medium employed improves the stability of the rhodium catalyst, i.e., resistance to catalyst precipitation, especially during the product recovery steps of the process. In these steps, distillation for the purpose of recovering the acetic acid product tends to remove from the catalyst the carbon monoxide, which in the environment maintained in the reaction vessel, is a ligand with a stabilizing effect on the rhodium.

Without being bound by theory, the rhodium component of the catalyst system is believed to be present in the form of a coordination compound of rhodium with a halogen component providing at least one of the ligands of such coordination compound. It is also believed that carbon monoxide will coordinate with rhodium. The rhodium component of the catalyst system may be provided by introducing into the reaction zone rhodium in the form of rhodium metal, rhodium salts such as the oxides, acetates, iodides, carbonates, hydroxides, chlorides, etc., or other compounds that result in the formation of a coordination compound of rhodium in the reaction environment.

The halogen-containing catalyst promoter of the catalyst system comprises a halogen compound, typically an organic halide. Thus, alkyl, aryl, and substituted alkyl or aryl halides can be used. Preferably, the halogen-containing catalyst promoter is present in the form of an alkyl halide. Even more preferably, the halogen-containing catalyst promoter is present in the form of an alkyl halide in which the alkyl radical corresponds to the alkyl radical of the feed alcohol, which is being carbonylated. Thus, in the carbonylation of methanol to acetic acid, the halide promoter may include a methyl halide, and more preferably methyl iodide.

A liquid reaction medium employed may include any solvent compatible with the catalyst system and may include pure alcohols, or mixtures of the alcohol feedstock and/or the desired carboxylic acid and/or esters of these two compounds. A preferred solvent and liquid reaction medium for the low water carbonylation process contains the desired carboxylic acid product. Thus, in the carbonylation of methanol to acetic acid, a preferred solvent system contains acetic acid.

The desired reaction rates may be obtained even at low water concentrations by maintaining in the reaction medium an ester of the desired carboxylic acid and an alcohol, desirably the alcohol used in the carbonylation, and an additional iodide ion that is over and above the iodide ion that is present as hydrogen iodide. A desired ester is methyl acetate. The additional iodide ion is desirably an iodide salt, with lithium iodide being preferred. Under low water concentrations, methyl acetate and lithium iodide act as rate promoters only when relatively high concentrations of each of these components are present and the promotion is higher when both of these components are present simultaneously. See, e.g., U.S. Pat. No. 5,001,259, incorporated herein by reference in its entirety. The absolute concentration of iodide ions is not a limitation on the usefulness of the present invention.

The carbonylation reaction of methanol to acetic acid product may be carried out by contacting the methanol feed with gaseous carbon monoxide bubbled through an acetic acid solvent reaction medium containing the rhodium catalyst, methyl iodide promoter, methyl acetate, and additional soluble iodide salt, at conditions of temperature and pressure suitable to form the carbonylation product. It will be generally recognized that it is the concentration of iodide ion in the catalyst system that is important and not the cation associated with the iodide ion, and that at a given molar concentration of iodide ion, the nature of the cation is not as significant as the effect of the iodide ion concentration. Any metal iodide salt, or any iodide salt of any organic cation, or quaternary cation such as a quaternary amine or phosphine or inorganic cation can be maintained in the reaction medium provided that the salt is sufficiently soluble in the reaction medium to provide the desired concentration of iodide ions. When the iodide salt is a metal salt, preferably it is an iodide salt of a member of the group consisting of the metals of Group IA and Group IIA of the periodic table as set forth in the “Handbook of Chemistry and Physics” published by CRC Press, Cleveland, Ohio, 2002 March (83rd edition). In particular, alkali metal iodides are useful, with lithium iodide being particularly suitable. In the low water carbonylation process most useful in this invention, the additional iodide ion over and above the iodide ion present as hydrogen iodide is generally present in the catalyst solution in amounts such that the total iodide ion concentration is from about 2 to about 20 wt. % and the methyl acetate is generally present in amounts of from about 0.5 to about 30 wt. %, and the methyl iodide is generally present in amounts of from about 5 to about 20 wt. %. The rhodium catalyst is generally present in amounts of from about 200 to about 2000 parts per million (ppm).

Typical reaction temperatures for carbonylation will be from 150 to 250° C., with the temperature range of 180 to 220° C. being a preferred range. The carbon monoxide partial pressure in the reactor can vary widely but is typically about 200 to about 3000 kPa, and preferably, about 300 to about 1000 kPa. Because of the partial pressure of by-products and the vapor pressure of the contained liquids, the total reactor pressure will range from about 1500 to about 4000 kPa.

In the carbonylation of methanol, permanganate reducing compounds (PRC's) such as acetaldehyde and PRC precursors may be formed as a byproduct, and as a result, the carbonylation system preferably includes a PRC Removal System (PRS) for removing such PRC's. PRC's may include, for example, compounds such as acetaldehyde, acetone, methyl ethyl ketone, butyraldehyde, crotonaldehyde, 2-ethyl crotonaldehyde, 2-ethyl butyraldehyde and the like, and the aldol condensation products thereof. Thus, in some embodiments, the invention relates to processes for reducing and/or removing PRC's or their precursors from intermediate streams during the formation of acetic acid by said carbonylation processes.

In particular, the present invention relates to a process in which a condensed light phase from a light ends column overhead is subjected to a distillation step to obtain an overhead that is subjected to a water extraction step to selectively reduce and/or remove PRC's from the process. In one embodiment, the distillation step in the PRS includes a single distillation column as described in U.S. Pat. No. 7,855,306, the entirety of which is incorporated herein by reference, while in other embodiments the distillation step may include two or more distillation steps as described, for example, in U.S. Pat. No. 6,143,930, the entirety of which is incorporated herein by reference. Similarly, in one embodiment, the extraction step in the PRS includes a single extraction unit, while in other embodiments, multiple extraction units employing the same or different extractants, may be employed, as described for example, in U.S. Pat. No. 7,223,886, the entirety of which is incorporated herein by reference. The principles of the invention may be employed with separation systems having single or multiple distillation columns and/or single or multiple extraction units.

The carbonylation reactor is typically either a stirred vessel or bubble-column type within which the reacting liquid or slurry contents are maintained automatically at a constant level. Into this reactor, fresh methanol, carbon monoxide and sufficient water as needed to maintain at least a finite concentration of water in the reaction medium are continuously introduced. Also introduced into the reactor is a recycled catalyst solution, such as from the flasher base, a recycled methyl iodide phase, a recycled methyl acetate phase, and a recycled aqueous acetic acid phase. A recycled phase may contain one or more of the foregoing components.

Distillation systems are employed that provide means for recovering the crude acetic acid and recycling catalyst solution, methyl iodide, methyl acetate, and other system components within the process. In a typical carbonylation process, carbon monoxide is continuously introduced into the carbonylation reactor, desirably below the agitator, which is used to stir the contents. The gaseous feed is thoroughly dispersed through the reacting liquid by this stifling means. A gaseous purge stream is desirably vented from the reactor to prevent buildup of gaseous byproducts and to maintain a set carbon monoxide partial pressure at a given total reactor pressure. The temperature of the reactor is controlled and the carbon monoxide feed is introduced at a rate sufficient to maintain the desired total reactor pressure.

Liquid product is drawn off from the carbonylation reactor at a rate sufficient to maintain a constant level therein and is introduced to the flasher. In the flasher, a catalyst-containing solution (catalyst phase) is withdrawn as a base stream (predominantly acetic acid containing the rhodium and the iodide salt along with lesser quantities of methyl acetate, methyl iodide, and water), while a vapor overhead stream comprising acetic acid is withdrawn overhead. The vapor overhead stream comprising acetic acid also contains methyl iodide, methyl acetate, and water. Dissolved gases exiting the reactor and entering the flasher comprise a portion of the carbon monoxide and may also contain gaseous by-products such as methane, hydrogen, and carbon dioxide. Such dissolved gases exit the flasher as part of the overhead stream.

### Acetic Acid to Ethanol

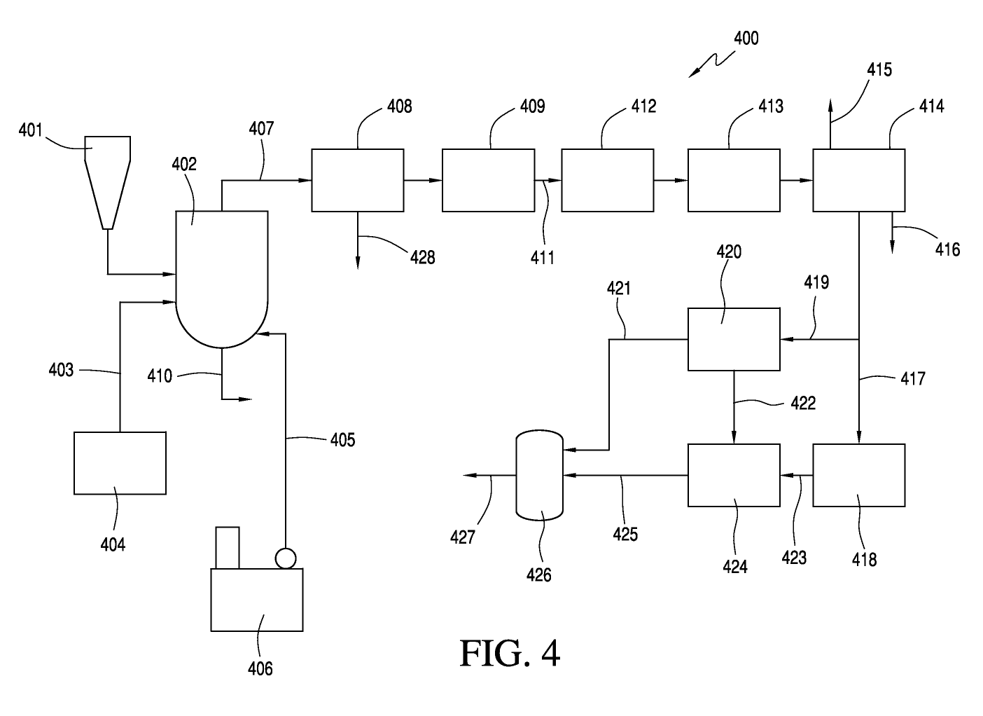

The process of the present invention may be used with any hydrogenation or hydrogenolysis process for producing ethanol. As shown in FIG. 4, there is provided an integrated process 400 for converting biomass 401 to ethanol. The pre-treatment steps for biomass 401 are not shown in FIG. 4. However, it should be understood that drying and pulverizing may be used as desired. Biomass 401 may be fed using a suitable feeding system to gasifier 402. In addition, an oxygen stream 403 from an air separation unit 404 may be fed to gasifier 402. Steam 405 may be produced from boiler 406, which preferably is powered by a renewable source such as wood, and supplied to gasifier 402. Preferably, gasifier 402 is a pressurized gasifier that operates at a pressure of at least 10 bar, at least 12 bar or at least 15 bar. Ash may be removed from gasifier in line 410 and may be recovered, for example, for use in fertilizer or cement applications. The product gas in line 407 is fed to tar removal vessel 408 and then cooled by cooler 409. Tar is removed in line 428. The cooled product gas in line 411 is fed with steam, preferably from a wood-fired boiler 406, to water gas shift reactor (WGSR) 412 to increase the hydrogen concentration of the syngas. In some embodiments, water derived from an ethanol reaction may be fed to the WGSR 412 and/or to the boiler 406 to provide water for steam. The pressure of the product gas is increased, if necessary, by compressor 413, preferably in less than 2 compression stages or less than 1 compression stage and at a compression ratio of less than 5:1, less than 3:1 or less than 2:1, followed by removing acidic gases in acid gas removal vessel 414. A carbon dioxide stream 415 is separated and purged from vessel 414. The carbon dioxide stream 415 may be used to dry the biomass during biomass pre-treatment. A sulfur containing purge stream 416 may also be purged. A portion of the syngas in line 417 may be fed to a methanol synthesis reactor 418 to form methanol. Another portion of the syngas in line 419 is fed to a cryogenic separator 420 and separated into a hydrogen stream 421 and a carbon monoxide stream 422. Methanol in line 423 and carbon monoxide stream 422 are fed to an acetic acid carbonylation process 424 to produce acetic acid in line 425. The acetic acid in line 425 is next fed to a hydrogenation reactor along with hydrogen stream 421 to form ethanol in line 427.

The materials, catalysts, reaction conditions, and separation processes that may be used in the hydrogenation of acetic acid to form ethanol are described further below.

Acetic acid, preferably obtained from the acetic acid unit described above, may also comprise other carboxylic acids and anhydrides, as well as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the ethanol synthesis reaction zones of the present invention without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen, optionally separated from the biomass-derived syngas, and/or recycle gas through the acetic acid at a temperature at or below 125° C., followed by heating of the combined gaseous stream to the reactor inlet temperature.

Some embodiments of the process of hydrogenating acetic acid to form ethanol may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation reaction may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected. Although there is no bar to the use of higher pressures, a considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, from 0.1 to 100 seconds, e.g., from 0.3 to 80 seconds or from 0.4 to 30 seconds.

The hydrogenation of acetic acid to form ethanol is preferably conducted in the presence of a hydrogenation catalyst. Suitable hydrogenation catalysts include catalysts comprising a first metal and optionally one or more of a second metal, a third metal or any number of additional metals, optionally on a catalyst support. The first and optional second and third metals may be selected from Group IB, IIB, IIIB, IVB, VB, VIIB, VIIB, VIII transition metals, a lanthanide metal, an actinide metal or a metal selected from any of Groups IIIA, IVA, VA, and VIA. Preferred metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron. Exemplary catalysts are further described in U.S. Pat. No. 7,608,744 and US Pub. No. 2010/0029995, the entireties of which are incorporated herein by reference. In another embodiment, the catalyst comprises a Co/Mo/S catalyst of the type described in US Pub. No. 2009/0069609, the entirety of which is incorporated herein by reference.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is selected from tin and rhenium.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 5 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 4 wt. %, e.g., from 0.1 to 3 wt. %, or from 0.1 to 2 wt. %.

In addition to one or more metals, in some embodiments of the present invention the catalysts further comprise a support or a modified support. As used herein, the term “modified support” refers to a support that includes a support material and a support modifier, which adjusts the acidity of the support material.

The total weight of the support or modified support, based on the total weight of the catalyst, preferably is from 75 to 99.9 wt. %, e.g., from 78 to 97 wt. %, or from 80 to 95 wt. %. In preferred embodiments that utilize a modified support, the support modifier is present in an amount from 0.1 to 50 wt. %, e.g., from 0.2 to 25 wt. %, from 0.5 to 15 wt. %, or from 1 to 8 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of ethanol.

Suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, and Bi₂O₃.

In another embodiment, the support modifier may be a basic modifier that has a low volatility or no volatility. Such basic modifiers, for example, may be selected from the group consisting of: (i) alkaline earth oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and US Pub. No. 2010/0197485 referred to above, the entireties of which are incorporated herein by reference.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a mole percentage based on acetic acid in the feed. The conversion may be at least 10%, e.g., at least 20%, at least 40%, at least 50%, at least 60%, at least 70% or at least 80%. Although catalysts that have high conversions are desirable, such as at least 80% or at least 90%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethyl oxygenates is at least 60%, e.g., at least 70%, or at least 80%. As used herein, the term “ethyl oxygenates” refers specifically to the compounds ethanol, acetaldehyde, and ethyl acetate. Preferably, the selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

Ethanol may be recovered using a variety of methods such as those described in US Pub. No. 2011/0190547, 2011/0190548, and 2011/0082322, the entire contents and disclosure of which is hereby incorporated by reference.

The ethanol product may be further treated. In one embodiment, the ethanol may be dehydrated using a water separation unit. The separation unit may be a dehydrating unit such as an extractive distillation column, membrane system, molecular sieve, azeotropic distillation, membrane pervaporation, and others. The ethanol may be anhydrous, preferably having a water content of less than 1 wt. %, e.g., less than 0.5 wt. %, less than 0.1 wt. %, less than 0.01 wt. %, less than 0.001 wt. % or less than 0.0001 wt. % based on the total weight of the substantially anhydrous ethanol composition.

The anhydrous ethanol composition may be suitable for use in a variety of applications, including fuels, solvents, chemical feedstocks, pharmaceutical products, cleansers, sanitizers, or hydrogenation transport. When the anhydrous ethanol feedstock is used as a chemical feedstock, it may be used to make other chemicals such as vinegar, ethyl acetate, ethylene, glycol ethers, ethylamines, aldehydes, and higher alcohols, especially butanol.

In one embodiment, the present invention may be useful for high yields of ethanol. In terms of gallons of ethanol per dry ton of biomass, the present invention may achieve ethanol yield that are greater than 90 gallons per dry ton of biomass, e.g., greater 110 or greater 130.

### Reduction of Acetic Acid to Ethyl Acetate

In some embodiments, the hydrogenation of acetic acid may favor the production of ethyl acetate over ethanol. U.S. Pat. No. 7,820,852, the entire disclosure of which is hereby incorporated by reference, described bimetallic catalyst that contain nickel, platinum, and/or palladium and at least one metal selected from copper and cobalt supported on a catalyst support selected from the group consisting of H-ZSM-5, silica, alumina, silica-alumina, calcium silicate, carbon, and mixtures. More particularly, the process for the selective formation of ethyl acetate from acetic acid may comprise hydrogenating acetic acid in the presence of hydrogen over a hydrogenating catalyst comprising at least one metal selected from the group consisting of nickel, platinum and palladium and at least one metal selected from molybdenum, rhenium, zirconium, copper and cobalt with the proviso that platinum may be used without molybdenum, rhenium, zirconium, copper or cobalt. In addition, the catalyst may be comprised of a suitable catalyst support optionally including one or more metal catalysts selected from the group consisting of ruthenium, iridium, chromium, tin, tungsten, vanadium and zinc. More specifically, the catalyst suitable for the process of this invention is typically comprised of a combination of platinum and copper supported on a suitable catalyst support or palladium and cobalt supported on a suitable catalyst support. Suitable catalyst supports include without any limitation, silica, alumina, calcium silicate, carbon, zirconia, zirconia-silica, titania, titania-silica, iron oxide and zeolite catalysts such as for example H-ZSM-5.

### Acetic Acid and Ethanol to Ethyl Acetate

In one embodiment, the acetic acid produced from methanol and the ethanol produced from acetic acid may be combined and esterified through an esterification reaction. Esterification may be carried out in either the liquid or vapor phase. The process may be operated continuously or batchwise.

Similar to methyl acetate, the formation of the esterification product in the esterification equilibrium reaction may be enhanced by the presence of a catalyst. A variety of homogeneous or heterogeneous acid catalysts may also be employed within the scope of this invention. The catalyst should be stable at the desired reaction temperature. Suitable catalysts include, without limitation, sulfuric acid, sulfonic acid, alkyl sulfonic acids, and aromatic sulfonic acids. Alkyl sulfonic and aromatic sulfonic acids may include methane sulfonic acid, benzene sulfonic acid and p-toluene sulfonic acid. In one embodiment, an ion exchange resin, e.g., Amberlyst™ 15, Amberlyst™ 36, Amberlyst™ 70, or Purolite™ CT179, may be used. Sulfuric acid, acidic zeolites, or heteropoly acids can also be used within the scope of the invention.

In either the vapor or liquid phase reactions, although ethanol and acetic acid may be fed in equimolar amounts, in some embodiments, ethanol may be employed in an excess molar amount in the reaction mixture. In another aspect, an excess of acetic acid is employed in the reaction mixture. For example, the molar ratio of acetic acid to ethanol optionally is greater than 1.01:1, e.g., greater than 1.05:1, greater than 1.2:1 or greater than 1.5:1. In terms of ranges, the molar ratio of acetic acid to ethanol may be from 1.01:1 to 5:1, e.g., from 1.01:1 to 3:1, from 1.05:1 to 3:1 or from 1.5:1 to 2.8:1. Without being bound by theory, the use of an excess molar amount of acetic acid, particularly under vapor phase esterification conditions, may reduce formation of diethyl ether. A molar ratio that is greater than 1.5:1 under vapor phase conditions, at reaction temperatures of less than 130° C., may result in substantially no formation of diethyl ether.

Additionally, the use of excess acetic acid may allow for higher conversion rates of ethanol in the esterification reactor. In one embodiment, at least 75% of the ethanol fed to the esterification reactor is converted to ethyl acetate, e.g., at least 90% or at least 95%.

The vapor-phase esterification reaction temperature is affected by the steady state composition and pressure, and typically may range from 50° C. to 200° C., e.g., from 80° C. to 190° C., from 125° C. to 175° C. The esterification process may be operated at atmospheric pressure but it is preferably operated at super-atmospheric pressure, e.g., from 5 to 700 kPa, from 30 to 350 kPa or from 100 to 300 kPa.

In another embodiment, the esterification may occur in the liquid phase in a distillation column. The esterification can be operated under batch or continuous mode. For commercial purposes, continuous operation is preferred. Suitable liquid phase esterification reactions are further described in U.S. Pat. Nos. 6,768,021, 6,765,110, and 4,481,146, the entire contents and disclosures of which are hereby incorporated by reference.

### Ethyl Acetate to Ethanol

Hydrogenolysis processes may also be integrated with the biomass gasifier according to one embodiment of the present invention. Hydrogenolysis involves the conversion of ethyl acetate to ethanol in the presence of hydrogen and a catalyst. Ethyl acetate may be obtained by esterification of acetic acid or through the hydrogenation of acetic acid. In one embodiment, the molar ratio of hydrogen to ethyl acetate that is introduced into hydrogenolysis reactor 140 is greater than 2:1, e.g. greater than 4:1, or greater than 12:1. In terms of ranges the molar ratio may be from 2:1 to 100:1, e.g., 4:1 to 50:1, or from 12:1 to 20:1. Without being bound by theory, higher molar ratios of hydrogen to ethyl acetate, preferably from 8:1 to 20:1, are believed to result in high conversion and/or selectivity to ethanol. The hydrogen used in the hydrogenolysis process may be advantageously separated from the biomass-derived syngas.

The hydrogenolysis reactor may comprise any suitable type of reactor, such as a fixed bed reactor or a fluidized bed reactor. Hydrogenolysis reactions are exothermic and in many embodiments an adiabatic reactor may be used for the hydrogenolysis reactor. Adiabatic reactors have little or no need for internal plumbing through the reaction zone to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used.

In preferred embodiments, a catalyst is employed in a fixed bed reactor, e.g., in the shape of a pipe or tube, where the reactants, typically in vapor form, are passed over or through the catalyst. Other reactors, such as fluid or ebullient bed reactors, can be employed. In some instances, a hydrogenolysis catalyst may be used in conjunction with an inert material to regulate the pressure drop of the reactant stream through the catalyst bed and the contact time of the reactant compounds with the catalyst particles.

The hydrogenolysis process may be operated in a vapor phase, or a mixed vapor/liquid phase regime. The mixed vapor/liquid phase regime is where the reactant mixture, at the reactor conditions, is below the dew point temperature. The hydrogenolysis reaction may change from a mixed vapor/liquid phase to a fully vapor phase reaction, as the reaction proceeds down the reactor. The mixed phase hydrogenolysis may also be conducted in other types of reactors, or within a combination of different reactors, for example in a slurry or stirred tank reactor with, or without, external circulation and optionally operated as a cascade or stirred tank, a loop reactor or a Sulzer mixer-reactor. The hydrogenolysis process may be conducted in batch, semi-continuous, or continuous mode. For industrial purposes, continuous mode of operation is the most efficient.

In some embodiments, the hydrogenolysis reactor may comprise other types of reactors, such as spinning basket and buss loop, or heat exchanger reactors. A mixed vapor/liquid phase hydrogenolysis reaction can be conducted with co-flow or counterflow of the vapor, e.g., hydrogen, to the liquid, i.e. ester feed stream, in a bubble reactor. Trickle bed reactors may also be used.

In one embodiment, a heterogeneous catalyst is used in the hydrogenolysis reactor. The catalyst may be a copper-based catalyst. The copper-based catalyst may comprise copper chromite, copper and zinc, and/or copper-zinc-oxide. Other copper-based catalyst may include an MgO—SiO₂ support that is impregnated with copper. Mixed copper oxide based catalyst may include copper and a second metal selected from zinc, zirconium, manganese, and/or oxides thereof. In some embodiments, aluminum oxide may also be present in the catalyst. The presence of aluminum oxide is believed to increase the heavy alcohol, and/or ketone concentrations during the reduction of ethyl acetate due to the presence of acidic sites. In those embodiments, the catalyst may comprise a basic component, such as magnesium or calcium, to reduce the acidic sites or the aluminum oxide concentration may be very low, e.g., less than 0.1 wt. %. In some embodiments, the catalyst may be substantially free of aluminum oxide.

A suitable copper-based catalyst may comprises from 30 to 70 wt. % copper oxide, 15 to 45 wt. % zinc oxide, and/or 0.1 to 20 wt. % aluminum oxide. More preferably, a copper-based catalyst may comprises from 55 to 65 wt. % copper oxide, 25 to 35 wt. % zinc oxide, and/or 5 to 15 wt. % aluminum oxide. Preferably, the copper-based catalyst is supported on zinc oxide and preferably comprises from 20 to 40 wt. % of copper, in terms of the metal content.

In other embodiments, the catalyst employed in the hydrogenolysis reactor may be a Group VIII-based catalyst. Group VIII-based catalyst may comprise a Group VIII metal selected from the group consisting of iron, ruthenium, osmium, cobalt, rhodium, iridium, nickel, palladium, and platinum. In addition, there may be one or more secondary promoter metals selected from the group consisting of zinc, cobalt, tin, germanium, lead, rhenium, tungsten, molybdenum. Group VIII-based catalysts may advantageously be supported on any suitable support known to those skilled in the art; non-limiting examples of such supports include carbon, silica, titania, clays, aluminas, zinc oxide, zirconia and mixed oxides. Preferably, the palladium based catalyst is supported on carbon. In addition, the Group VIII-based catalyst may be supported on any suitable support, such as silica, silica-alumina, calcium metasilicate, carbon, titania, clays, aluminas, zinc oxide, zirconia, and mixed metal oxides.

The reduction of ethyl acetate to produce ethanol, e.g., in the hydrogenolysis reactor, is typically conducted at elevated temperatures from 125° C. to 350° C., e.g., from 180° C. to 345° C., from 225° C. to 310° C., or from 290° C. to 305° C. Reaction temperatures greater than 240° C., or greater than 260° C., may increase conversion of ethyl acetate. Although not bound by theory, it is believed that reduced temperatures in the hydrogenolysis reactor of less than 275° C. may suppress the formation of heavy impurities such as alcohols and/or ketones. The pressure in the hydrogenolysis reactor may operate under high pressure from greater than 1000 kPa, e.g., greater than 3,000 kPa or greater than 5,000 kPa. In terms of ranges the pressure in the hydrogenolysis reaction may be from 700 to 8,500 kPa, e.g., from 1,500 to 7,000 kPa, or from 2,000 to 6,500 kPa. Pressure greater than 2,500 kPa may be more favorable for improving ethanol productivity and/or selectivity. The reactants may be fed to hydrogenolysis reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 20,000 hr⁻¹, e.g., from 1000 hr⁻¹ to 10,000 hr⁻¹, or from 2000 hr⁻¹ to 7,000 hr⁻¹.

In particular, the reaction of ethyl acetate may achieve favorable conversion of ethyl acetate and favorable selectivity and productivity to ethanol. The conversion may be at least 50%, e.g., at least 70%, at least 90%. In terms of ranges, the conversion of ethyl acetate may range from 50 to 98%, e.g., from 60 to 95% or from 70 to 90%. Although catalysts and reaction conditions that have high conversions may be possible, such as greater than 90% or greater than 95%, in some embodiments a low conversion may be acceptable at high selectivity for ethanol. Compensating for low conversion by appropriate recycle streams or use of larger reactors may be easier than compensating for poor selectivity to ethanol.

Selectivity is expressed as a mole percent based on converted ethyl acetate. It should be understood that each compound converted from ethyl acetate has an independent selectivity and that selectivity is independent from conversion. For example, if 90 mole % of the converted ethyl acetate is converted to ethanol, we refer to the ethanol selectivity as 90%. The selectivity to ethanol is preferably at least 80%, e.g., at least 90% or at least 95%.

A productivity of at least 100 grams of ethanol per kilogram of catalyst per hour, e.g., at least 500 grams of ethanol per kilogram of catalyst per hour or at least 1,000 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

### Ethanol to Ethylene

In one embodiment, the anhydrous ethanol composition, formed ultimately from biomass-derived syngas, may be dehydrated to produce renewable ethylene. Any known dehydration catalysts can be employed, such as those described in copending US Pub. 2010/0030001 and 2010/0030002, the entire contents and disclosures of which are hereby incorporated by reference. A zeolite catalyst, for example, may be employed as the dehydration catalyst. Preferably, the zeolite has a pore diameter of at least about 0.6 nm and preferred zeolites include dehydration catalysts selected from the group consisting of mordenites, ZSM-5, a zeolite X and a zeolite Y.

Known dehydration catalysts can be employed in the second step of the process of this invention. Typically, a zeolite catalyst is employed as a dehydration catalyst. While any zeolite having a pore diameter of at least about 0.6 nm can be used, preferably employed among such zeolites are the dehydration catalyst selected from the group consisting of mordenites, ZSM-5, a zeolite X and a zeolite Y. The preparation of large-pore mordenites is described, for example, in U.S. Pat. No. 4,018,514. Zeolite X is described, for example, in U.S. Pat. No. 2,882,244 and zeolite Y in U.S. Pat. No. 3,130,007, the entireties of which are hereby incorporated by reference. Various zeolites and zeolite-type materials are known in the art for the catalysis of chemical reactions. For example, U.S. Pat. No. 3,702,886 discloses a class of synthetic zeolites, characterized as “Zeolite ZSM-5”, the entirety of which is hereby incorporated by reference, which is effective for the catalysis of various hydrocarbon conversion processes. The zeolites suitable for the procedure of the invention can be in the basic form, in the partially or totally acidified form, or in the partially dealuminated form.

The active catalyst, characterized as “H-ZSM-5” or “H-mordenite” zeolites are prepared from a corresponding “ZSM-5” zeolite or “mordenite” zeolite by replacing most, and generally at least about 80% of the cations of the latter zeolite with hydrogen ions using techniques well-known in the art. These zeolite catalysts are essentially crystalline aluminosilicates or in the neutral form a combination of silica and alumina in a well defined crystalline structure. In a particularly preferred class of zeolite catalysts for purposes of the present invention, the molar ratio of SiO₂ to Al₂O₃ in these zeolites is within the ratio of about 10 to 60.

### Acetic Acid and Ethylene to Vinyl Acetate

Ethylene, produced by dehydrating ethanol or via the MTO process, may be further reacted with acetic acid and oxygen to produce vinyl acetate (VA) as described, for example, in U.S. Pat. Nos. 7,885,303, and 7,820,853, the entire contents of which are hereby incorporated by reference. Typically, VA formation is carried out heterogeneously with the reactants being present in the gas phase. Molecular oxygen-containing gas used in formation of VA in embodiments of the process of the present invention may comprise other inert gases such as nitrogen. Preferably, molecular oxygen is used in the acetoxylation of ethylene to form VA instead of air. The reaction may suitably be carried out at a temperature in the range from 140° C. to 220° C. and at a pressure in the range from 100 to 10,000 kPa. The reaction can be carried out in any suitable reactor design capable of removing the heat of reaction in an appropriate way; preferred technical solutions are fixed or fluidized bed reactors as described herein.

Acetic acid conversions in the range of about 5 to 50% may be achieved. Oxygen conversions in the range of about 20 to 100% may also be achieved. The catalyst suitably has a productivity (space time yield, STY) in the range of 100 to 2000 grams of vinyl acetate per hour per liter of catalyst, but >10000 grams of vinyl acetate per hour per liter of catalyst is also suitable.

The gaseous product stream comprises VA and water and optionally also unreacted acetic acid, ethylene, ethyl acetate, ethane, nitrogen, carbon monoxide, carbon dioxide and possibly traces of other byproducts. Ethylene, ethane, carbon monoxide and carbon dioxide, if any, are preferably removed from the product stream in an intermediate step, suitably as an overhead gaseous fraction from a scrubbing column, in which a liquid fraction comprising vinyl acetate, water and acetic acid is removed from the base.

The product stream comprising VA, water and acetic acid, with or without the intermediate scrubbing step, is separated in the final step by distillation into an overhead azeotrope fraction comprising vinyl acetate and water and a base fraction comprising acetic acid.

VA is recovered from the azeotrope fraction, suitably for example by decantation. The recovered VA may, if desired, be further purified in a known manner. The base fraction comprising acetic acid is preferably recycled, with or preferably without further purification, to the acetic acid hydrogenation reactor.

### VA to PVAc, EVAc, PVOH, and EVOH

VA may be polymerized through well-known processes to make polyvinyl acetate (PVAc), or may be copolymerized in the presence of a comonomer, such as an alkene, e.g., ethylene, to form ethylene/vinyl acetate copolymer (EVAc). Polyvinyl acetate and ethylene-vinyl acetate copolymer may be further processed to form polyvinyl alcohol (PVOH) and ethylene-vinyl alcohol copolymer (EVOH), respectively. Thus, the production of PVOH or copolymers of PVOH from vinyl acetate involves two steps. The first step is the polymerization of vinyl acetate to form polyvinyl acetate, and the second step involves alcoholysis of the polyvinyl acetate to form PVOH. Exemplary processes for forming PVOH are described in U.S. Pat. Nos. 4,463,138; 4,820,767; 7,468,455 and U.S. Pat. No. 7,855,303, each of which is incorporated herein by reference in its entirety.

Polymerization of the vinyl acetate monomer is accomplished by initiating radical polymerization. Oxygen acts as an inhibitor of radical polymerization and, accordingly, the polymerization is preferably carried out under substantially oxygen free conditions. Thus, the fractionated highly pure vinyl acetate monomer is preferably subjected to deoxygenation procedures prior to polymerization. This may be accomplished by a freeze-thaw operation under a high vacuum and an inert gas sweep wherein the monomer is frozen at about −93° C., thawed, refrozen, thawed, etc. The vinyl acetate monomer may be subjected to at least about three freeze-thaw cycles in order to ensure an essentially complete removal of oxygen. However, removal of oxygen by bubbling pure nitrogen through the polymerization mixture may also be also adequate.

Once a purified and deoxygenated vinyl acetate monomer is obtained, the monomer may then be transferred to a suitable reactor for conducting the free radical bulk polymerization process. Reactors for use in the polymerizing reaction are not critical, and reactors used in conventional bulk polymerizations can be used. Suitable reactors will usually be equipped with a temperature control means to maintain the reaction mixture within the desired temperature range and should also be equipped with means to maintain the reactor substantially oxygen free; as for example, means for carrying out the polymerization under an inert gas such as nitrogen.

The polymerization process can be conducted in a batch, semicontinuous or continuous fashion. The reaction can be conducted in a single reaction zone or in a plurality of reaction zones, in series or in parallel, or it may be conducted intermittently or continuously in an elongated tubular zone or series of such zones. The materials of construction employed should be inert to the reactants during the reaction and able to withstand the reaction temperatures and pressures.

The reaction zone can be fitted with one or more internal and/or external heat exchanger(s) in order to control undue temperature fluctuations, or to prevent any possible runaway reaction temperatures or fluctuations therein. In preferred embodiments of the process, agitation means to vary the degree of mixing of the reaction mixture can be employed. Mixing by vibration, shaking, stirring, rotation, oscillation, ultrasonic vibration or the like are all illustrative of the type of agitation means contemplated. Such means are available and well known to those skilled in the art.

Upon completion of the polymerization process, unreacted vinyl acetate may be removed by distillation under atmospheric or sub-atmospheric pressures. A polymeric residue comprising polyvinyl acetate will remain in the vessel utilized for the removal of unreacted vinyl acetate. The polyvinyl acetate product may be worked up by conventional means, as for example by initially dissolving the polymeric residue in an organic solvent such as acetone, tetrahydrofuran, methanol, dichloromethane, ethyl acetate, etc., and then precipitating the polymer with a non-solvent such as hexane, cyclohexanol, diethyl ether, mesitylene or the like. Similarly, precipitation of the polymers may be accomplished by simply employing cold water. Recovery of the polymer is then accomplished by standard filtration and drying procedures.

Polyvinyl acetate produced by the above process will have an intrinsic viscosity, and thus a corresponding molecular weight which is only slightly higher than reacetylated polyvinyl acetate produced from PVOH resulting from alcoholysis of the original polyvinyl acetate. Thus, the polyvinyl acetate that is produced may be essentially linear. Polyvinyl acetate produced in accordance with this process may have an intrinsic viscosity that is equal to or greater than about 3.2 dL/g. This corresponds to a weight average molecular weight of equal to or greater than about 1.0×10⁶. Thus, given the fact that the repeat unit of PVAc has a molecular weight of about 86 and the repeating unit of PVOH has a molecular weight of about 44, PVOH produced by the alcoholysis of such polyvinyl acetate has a weight average molecular weight of at least about 0.45×10⁶. In a preferred embodiment of the invention, the polyvinyl acetate has an intrinsic viscosity ranging from about 3.5 dL/g to about 4.0 dL/g. Polyvinyl acetate falling within this intrinsic viscosity range has a weight average molecular weight ranging from about 1.3×10⁶ to about 1.6×10⁶, and PVOH prepared by the alcoholysis of this material will have a weight average molecular weight ranging from about 0.5×10⁶ to about 0.8×10⁶.

In the second step, the alcoholysis may be accomplished by initially dissolving the PVAc (or EVAc) in a quantity of a low molecular weight alcohol such as methanol sufficient to form at least about a 2% solution of the polyvinyl acetate resin. Base or acid catalysis may then be employed in order to convert the PVAc to PVOH which is produced in the form of a gel. Base catalysis is preferred, however, with suitable bases including anhydrous potassium hydroxide, anhydrous sodium hydroxide, sodium methoxide, potassium methoxide, etc. The alcoholysis reaction may take place under anhydrous or substantially anhydrous conditions, for example, when sodium hydroxide is used as the base, to avoid unwanted formation of sodium acetate instead of the desired methyl acetate. The gel material is optionally chopped into small pieces and may be extracted repeatedly with methanol, ethanol or water for removal of residual base salts. The essentially pure PVOH may be dried under vacuum at a temperature of about 30° C. to about 70° C. for about 2 to 20 hours. PVOH produced in accordance with the process may have a weight average molecular weight of at least about 0.45×10⁶. In a preferred embodiment, the weight average molecular weight of the PVOH is from about 0.45×10⁶ to about 1.0×10⁶, e.g., from about 0.5×10⁶ to about 0.8×10⁶.

PVOH produced in accordance with this invention may be useful in the production of PVOH fibers of excellent strength. Also, fibers produced from the PVOH of this invention preferably have high melting points.

The above-described alcoholysis reaction may be similarly employed in the formation of copolymers of polyvinyl alcohol, and in particular, in the alcoholysis of ethylene/vinyl acetate copolymer to form EVOH.

Although the biomass gasifier is shown integrated with several types of chemical processes, as exemplified in FIG. 1, the biomass gasifier may be advantageously integrated with high pressure industrial chemical processes that require high pressure syngas.

While the invention has been described in detail, modifications within the spirit and scope of the invention will be readily apparent to those of skill in the art. In addition, it should be understood that aspects of the invention and portions of various embodiments and various features recited herein and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing syngas, comprising:
(a) introducing aquatic biomass, a fossil fuel, water and oxygen to a gasifier and forming syngas comprising hydrogen, carbon monoxide and carbon dioxide; and
(b) feeding aquatic biomass with carbon dioxide derived from the syngas.

2. The process of claim 1, wherein the aquatic biomass that is introduced into the gasifier comprises the aquatic biomass that is fed in step (b).

3. The process of claim 1, wherein the aquatic biomass is selected from the group consisting of microalgae, macroalgae, microplants, duckweed, water hyacinth, cattails, banana tree stem, kelp, and green algae.

4. The process of claim 1, wherein the gasifier is an entrained flow slagging gasifier.

5. The process of claim 4, wherein the gasifier is operated at a pressure greater than 30 bar.

6. The process of claim 4, wherein runoff from the gasifier provides nutrients for the aquatic biomass that is fed with carbon dioxide.

7. The process of claim 4, wherein the aquatic biomass and fossil fuel are introduced to the gasifier at a weight ratio from 1:99 to 40:60.

8. The process of claim 4, wherein the aquatic biomass is formed from microalgae having an average size less than 15 μm.

9. The process of claim 4, wherein the aquatic biomass is formed from macroalgae having an average size greater than 15 μm.

10. The process of claim 4, wherein the fossil fuel comprises coal or natural gas.

11. A process for producing ethanol from aquatic biomass and a fossil fuel, comprising the steps of:
(a) introducing aquatic biomass, a fossil fuel, water and oxygen to a gasifier and forming syngas comprising hydrogen, carbon monoxide and carbon dioxide; and
(b) feeding aquatic biomass with carbon dioxide derived from the syngas.
(c) compressing the syngas to form compressed product gas;
(d) directing a first portion of the compressed product gas to an alcohol synthesis reactor to produce methanol;
(e) directing a second portion of the compressed product gas to a gas separator to produce a hydrogen stream and a carbon monoxide stream;
(f) reacting the carbon monoxide stream with the methanol to produce acetic acid; and
(g) reacting the hydrogen stream with acetic acid to produce ethanol.

12. The process of claim 11, wherein the compressing step occurs at a compression ratio less than 5:1.

13. A process for producing syngas, comprising:
(a) introducing aquatic biomass, a fossil fuel, water and oxygen to a gasifier and forming a product gas comprising hydrogen and carbon monoxide;
(b) increasing the ratio of hydrogen to carbon monoxide using the water gas shift reaction and forming byproduct carbon dioxide; and
(c) feeding aquatic biomass with carbon dioxide formed in step (b).

14. The process of claim 13, wherein the gasifier is an entrained flow slagging gasifier.

15. The process of claim 13, wherein the aquatic biomass is selected from the group consisting of microalgae, macroalgae, microplants, duckweed, water hyacinth, cattails, banana tree stem, kelp, and green algae.

16. The process of claim 15, wherein the aquatic biomass is selected from the group consisting of microalgae, macroalgae, microplants, duckweed, and water hyacinth.

17. The process of claim 15, wherein the gasifier is operated at a pressure greater than 30 bar.

18. The process of claim 15, wherein runoff from the gasifier provides nutrients for the algae.

19. The process of claim 15, wherein the aquatic biomass and fossil fuel are introduced to the gasifier at a weight ratio from 1:99 to 40:60.

20. The process of claim 15, wherein the aquatic biomass is formed from microalgae having an average size less than 15 μm.

21. The process of claim 15, wherein the aquatic biomass is formed from macroalgae having an average size greater than 15 μm.

22. The process of claim 15, wherein the fossil fuel comprises coal or natural gas.
